(19) United States
(12) Patent Application Publication
(10) Pub. No.: US 2005/0154535 A1
(43) Pub. Date: Jul. 14, 2005

(54) Method, system and apparatus for assembling and using biological knowledge

(75) Inventors: Justin Sun, Norwood, MA (US); D. Navin Chandra, Framing-ham, MA (US); Dexter R. Pratt, Reading, MA (US); David A. Kightley, York, ME (US); Joshua Levy, Chapel Hill, NC (US); Maria Fatima Chandra, Framing-ham, MA (US)

Correspondence Address: TESTA, HURWITZ & THIBEAULT, LLP, HIGH STREET TOWER, 125 HIGH STREET, BOSTON, MA, 02110, US

(73) Assignee: Genstruct, Inc., Cambridge, MA (US)

(21) Appl. No.: 10/794,407

(22) Filed: Mar. 5, 2004

Related U.S. Application Data
(60) Provisional application No. 60/535,352, filed on Jan. 9, 2004.

(51) Int. Cl. G06F 19/00; G01N 33/48; G01N 33/50

(52) U.S. Cl. 702/19

(57) ABSTRACT

Disclosed are methods, systems and apparatus for constructing assemblies of biological knowledge constituting a biological knowledge base, and for subsetting and transforming life sciences-related data and information into biological models to facilitate computation and electronic reasoning on biological information. A subset of data is extracted from a global knowledge base or repository to reconstruct a more specialized sub-knowledge base or assembly designed specifically for the purpose at hand. Assemblies generated by the invention permit selection and rational organization of seemingly diverse data into a model of any selected biological system, as defined by any desired biological criteria. These assemblies can be mined easily and can be logically reasoned with great productivity and efficiency.

### RELATED APPLICATIONS

This application claims the benefit of U.S. provisional application No. 60/535,352, entitled “Method, System And Apparatus for Assembling and Using Biological Knowledge,” filed Jan. 9, 2004, the disclosure of which is incorporated by reference herein.

### TECHNICAL FIELD

The invention relates to methods, systems and apparatus for discovering new biological knowledge, and more particularly, to methods, systems and apparatus for assembling a biological knowledge base, to methods, systems and apparatus for subsetting and transforming life sciences-related data and information into biological models, and to methods, systems and apparatus to facilitate computation and electronic reasoning on biological information.

### BACKGROUND

The amount of biological information generated in the today's world is increasing dramatically. It is estimated that the amount of information now doubles every four to five years. Because of the large amount of information that must be processed and analyzed, traditional methods of discerning and understanding the meaning of information, especially in the life science-related areas, are breaking down.

To form an effective understanding of a biological system, a life science researcher must synthesize information from many sources. Understanding biological systems is made more difficult by the interdisciplinary nature of the life sciences. Forming an understanding of a biological system may require in-depth knowledge of genetics, cell biology, biochemistry, medicine, and many other fields. Understanding a system may require that information of many different types be combined. Life science information may include material on basic chemistry, proteins, cells, tissues, and effects on organisms or population—all of which may be interrelated. These interrelations may be complex, poorly understood, or hidden.

There are ongoing attempts to produce electronic models of biological systems. These involve compilation and organization of enormous amounts of data, and construction of a system that can operate on the data to simulate the behavior of a biological system. Because of the complexity of biology, and the sheer numbers of data, the construction of such a system can take hundreds of man years and multiple tens of millions of dollars. Furthermore, those seeking new insights and new knowledge in the life sciences are presented with the ever more difficult task of connecting the right data from mountains of information gleaned from vastly different sources. Companies willing to invest such resources so far have been unsuccessful in compiling models of real utility which aid researchers significantly in advancing biological knowledge. Thus, to the extent current systems of generating and recording life science data have been developed to permit knowledge processing and analysis, they are clearly far from optimal, and significant new efficiencies are needed.

More specifically, what is needed in the art is a way to assemble vast amounts of diverse life science-related knowledge, and to produce from it insightful and meaningful models that can be probed and queried to discern new biological relationships, pathways, causes and effects, and other insights with efficiency and ease.

### SUMMARY OF THE INVENTION

In accordance with the invention, it has been realized that a key to providing useful and manageable biological knowledge bases that are capable of effectively modeling biological systems is to provide means for rapidly and efficiently building sub-knowledge bases and derived knowledge bases. These specialty knowledge bases can be constructed from a global knowledge base by extracting a potentially relevant subset of life science-related data satisfying criteria specified by a user as a starting point, and reassembling a specially focused knowledge base having the structure disclosed herein. These can be refined, augmented, probed, displayed in various formats, and mined using human observation and analysis and using a variety of tools to facilitate understanding and revelation of hidden interactions and relationships in biological systems, i.e., to produce new biological knowledge. This in turn permits the generation of new hypotheses concerning biological pathways based on the new biological knowledge, and permits the user to design and conduct biological experiments using biomolecules, cells, animal models, or a clinical trial to validate or refute a hypothesis.

The invention thus provides a novel paradigm, methods, apparatus, and tool set which can be applied to a global knowledge base. The tools and methods enable efficient execution of discovery projects in the life sciences-related fields. The invention provides new methods and tools which permit one to condition a knowledge base to facilitate both focus and flexibility in a project or task. The invention also permits one to address any biological topic, no matter how obscure or esoteric, provided there are at least some assertions in a global knowledge base relevant to the topic. Assertions represent facts relating existing objects in a system, or a fact about one object in the system and some literal value, or any combination thereof. Each fact within a knowledge base or assembly is referred to herein as an assertion.

One aspect of the present invention is to extract from a global knowledge base or repository a subset of data that is necessary or helpful and to reconstruct a more specialized sub-knowledge base designed specifically for the purpose at hand. In this respect, it is important that the structure of the global knowledge base be designed such that one can extract a sub-knowledge base that preserves relevant relationships between information in the sub-knowledge base. The sub-knowledge base, or what is referred to herein simply as an assembly, permits selection and rational organization of seemingly diverse data into a coherent model of any selected biological system, as defined by any desired combination of criteria. These assemblies are microcosms of the global knowledge base, can be more detailed and comprehensive than the global knowledge base in the area they address, and can be mined more easily and with greater productivity and efficiency. Assemblies can be merged with one another, used to augment one another or can be added back to the global knowledge base. As referred to herein, the terms assembly and knowledge base are meant to be interchangeable.

In an important aspect of the invention, the invention allows for the generation of derived assemblies. Derived assemblies are those in which new assertions are created based on logical inferences from other assertions. Derived assemblies can be augmented through reasoning and other algorithms. Augmentation is done by adding new knowledge that may or may not be part of the original assembly, or in the global knowledge base. Augmentation includes, but is not limited to, performing reasoning on the assembly and examining the assembly together with external data (e.g., laboratory data, clinical data, literature data).

The invention provides methods for assembling a knowledge base, the means for creating it, and the tools for refining it. In a particular aspect, the invention provides methods for assembling a biological knowledge base by first providing a database of biological assertions, or means, such as a user interface, for accessing such a knowledge base, comprising a multiplicity of nodes representative of biological elements and descriptors characterizing the elements or relationships among them. A preferred knowledge base is disclosed in co-pending, co-owned U.S. patent application Ser. No. 10/644,582, the disclosure of which is incorporated by reference herein. Next, the method extracts a subset of assertions from the knowledge base that satisfies a set of biological criteria specified by a user to define a selected biological system. The extracted data then are compiled to produce an assembly, i.e., a biological knowledge base of assertions potentially relevant to the selected biological system.

The invention provides methods for discovering new biological knowledge. The methods include providing a database of biological assertions comprising a multiplicity of nodes representative of biological elements and descriptors characterizing the elements or relationships among them. The methods also include extracting a subset of assertions from the database that satisfy a set of biological criteria specified by a user to define a selected biological system. The methods further include compiling the extracted assertions to produce a biological knowledge base of assertions potentially relevant to the selected biological system and then analyzing the biological knowledge base to discover new biological knowledge. The invention also provides methods for generating new biological knowledge by providing a database of biological assertions that include a multiplicity of nodes representative of biological elements and descriptors characterizing the elements or relationships among nodes, and then transforming a plurality of the biological assertions to produce a derived knowledge network.

The invention provides methods for mining a biological knowledge base including providing a database of biological assertions that have a multiplicity of nodes representative of biological elements and descriptors characterizing the elements or relationships among nodes, transforming a plurality of the biological assertions to produce a derived knowledge network, and mining the assembly to discover new biological knowledge.

The invention provides systems for assembling a biological knowledge base. The systems include a database of biological assertions in electronic format comprising a multiplicity of nodes representative of biological elements and descriptors characterizing the elements or relationships among them. The systems also include an application which functions to extract a subset of assertions from the database that satisfy a set of biological criteria specified by a user to define a selected biological system. The systems further include a knowledge assembler configured to compile the extracted assertions to produce a biological knowledge base of assertions potentially relevant to the selected biological system. The invention also provides systems for assembling a biological knowledge base including a database of biological assertions that have a multiplicity of nodes representative of biological elements and descriptors characterizing the elements or relationships among nodes, and an application to transform a plurality of biological assertions to produce a derived knowledge network.

The invention provides computing devices for assembling a biological knowledge base and for discovering new biological knowledge. The computing devices include means for accessing an electronic database of biological assertions comprising a multiplicity of nodes representative of biological elements and descriptors characterizing the elements or relationships among them, and a user interface for specifying biological criteria which will be used by the device for constructing an assembly constituting a selected biological system. The devices also include a computer application to extract a subset of assertions from the database that satisfy the biological criteria specified by a user, and a knowledge assembler configured to compile the extracted assertions to produce a biological knowledge base of assertions potentially relevant to the selected biological system. The invention also provides articles of manufacture having a computer-readable program carrier with computer-readable instructions embodied thereon for performing the methods and systems described above.

In various embodiments, the invention includes method steps, applications, and devices for applying reasoning to the extracted assertions to remove logical inconsistencies in the knowledge base; applying reasoning to the extracted assertions to generate new biological knowledge; applying reasoning to the extracted assertions to augment the assertions therein by adding to the knowledge base additional assertions from the database satisfying the selection criteria; or augmenting the assertions therein by adding to the knowledge base additional assertions from data sources extraneous to the database.

In various embodiments, the invention includes method steps, applications, and devices for applying reasoning to the extracted assertions to augment the assertions therein by: adding to the knowledge base additional assertions that are novel to the assembly; applying pathway analysis to the knowledge assembly to extract one or more pathways that relates to experimental data or clinical data; applying homology transformation to the extracted assertions; applying logical simulation to the extracted assertions; or adding to the assembly additional assertions from data sources extraneous to the database.

In various embodiments, the invention includes method steps, applications, and devices for inferring new assertions from the biological assertions; extracting a subset of assertions from the database that satisfy a set of biological criteria specified by a user to define a selected biological system; performing mathematical operations on sets of biological assertions to produce new sets of assertions; and summarizing biological assertions to produce new assertions.

In various embodiments, nodes are enzymes, cofactors, enzyme substrates, enzyme inhibitors, DNAs, RNAs, transcription regulators, DNA activators, DNA repressors, signaling molecules, trans membrane molecules, transport molecules, sequestering molecules, regulatory molecules, hormones, cytokines, chemokines, antibodies, structural molecules, metabolites, vitamins, toxins, nutrients, minerals, agonists, antagonists, ligands, receptors, or combinations thereof. In other embodiments, nodes are protons, gas molecules, organic molecules, amino acids, peptides, protein domains, proteins, glycoproteins, nucleotides, oligonucleotides, polysaccharides, lipids, glycolipids, or combinations thereof. In further embodiments, nodes comprise cells, tissues, or organs, or drug candidate molecules.

In various embodiments, biological information represented by nodes and assertions may include experimental data, knowledge from the literature, patient data, clinical trial data, compliance data; chemical data, medical data, or hypothesized data. In other embodiments, biological information may represent facts about of a molecule, biological structure, physiological condition, trait, phenotype, or biological process.

In various embodiments, the biological information represents a molecule, biological structure, physiological condition, trait, phenotype, biological process, clinical data, medical data, disease data or chemistry. In some embodiments, the biological information includes a descriptor of the condition, location, amount, or substructure of a molecule, biological structure, physiological condition, trait, phenotype, biological process, clinical data, medical data, disease data or chemistry.

In various embodiments, the new biological knowledge produced by the method includes predictions of physiological behavior in humans, for example, from analysis of experiments conducted on animals, such as drug efficacy and/or toxicity, or the discovery of biomarkers indicative of the prognosis, diagnosis, drug susceptibility, drug toxicity, severity, or stage of disease. In some embodiments, the method includes comparing different assemblies; in others, mapping data, and in still others, graphically presenting all or various portions of the assembly so as to facilitate human understanding, extrapolation, interpolation, and reasoning.

The foregoing and other features and advantages of the present invention, as well as the invention itself, will be more fully understood from the description, drawings, and claims which follow.

### BRIEF DESCRIPTION OF THE DRAWINGS

In the drawings, like reference characters generally refer to the same parts throughout the different views. The drawings are not necessarily to scale, emphasis instead generally being placed upon illustrating the principles of the invention. In the following description, various embodiments of the invention are described with reference to the following drawings, in which:

FIG. 1 is an overview diagram showing an illustrative embodiment of the invention.

FIG. 2A shows an original network and FIG. 2B shows a subset of a network in accordance with an illustrative embodiment of the invention.

FIG. 3 shows a knowledge assembly graph in accordance with an illustrative embodiment of the invention.

FIG. 4 shows the merger of two pathways in accordance with an illustrative embodiment of the invention.

FIG. 5 shows a knowledge assembly graph in accordance with an illustrative embodiment of the invention.

FIG. 6 shows a knowledge assembly graph in accordance with an illustrative embodiment of the invention.

FIG. 7 shows a transformed network in accordance with an illustrative embodiment of the invention.

FIG. 8 shows a representation of a summarized metabolic reaction in accordance with an illustrative embodiment of the invention.

FIG. 9 shows a derived network in accordance with an illustrative embodiment of the invention.

FIG. 10 shows an illustrative example of data mapping in accordance with an embodiment of the invention.

FIG. 11 shows inference paths for upstream causes starting with a change in mRNA levels for a particular gene in accordance with an illustrative embodiment of the invention.

FIG. 12 is a diagram showing propagation of predicted changes in a forward simulation being compared with observed expression changes in accordance with an illustrative embodiment of the invention.

FIG. 13 is a diagram generated by a backward simulation from nine expression data points, followed by pruning of the graph to show only the chains of reasoning which support the primary hypotheses, in accordance with an illustrative embodiment of the invention.

FIG. 14 shows an illustrative example of a visualization technique in accordance with the present invention that is based on a forward simulation that compares predicted outcomes with actual laboratory data.

FIG. 15 shows an assembly overview graph in accordance with an illustrative embodiment of the invention.

FIG. 16 is a graph showing simulation results in accordance with an illustrative embodiment of the invention.

FIG. 17 shows a visualization of time series expression and proteometric data mapped onto a segment of a known metabolic pathway in accordance with an illustrative embodiment of the invention.

FIG. 18 shows a diagram that indicates a means of summarizing time, dose, or other data series data from many experiments around a particular gene or protein in accordance with an illustrative embodiment of the invention.

FIG. 19 shows a pie chart that summarizes the correspondence of a hypothesis to observed data in accordance with an illustrative embodiment of the invention.

FIG. 20 shows an example of an algorithm for use in validating a biological model by comparing predicted to actual results in accordance with the invention.

FIG. 21 shows an example of a biomarker identification algorithm in accordance with the invention.

### DESCRIPTION

To implement the present invention, a global knowledge base, or central database, is structured to comprise a multiplicity of nodes and descriptors, and these nodes and descriptors can be copied or transferred without losing any internal consistency or biological context. Nodes are elements of biological systems, both physical and functional, and include such things, for example, as specific organs, tissues, cells, organelles, cell compartments, membranes, proteins, DNAs, RNAs, small molecules, drugs, and metabolites. The descriptors are data entries interrelating the nodes functionally and/or structurally (e.g., case frames, which are “verbs” identifying the interrelationship of nodes), and data entries associating additional information with either or both the nodes and their interrelationships (e.g., recording the species or organ where the protein is found, identifying the journal where the data were reported, notation of tertiary structural information about the subject protein, notation that the protein is elevated in patients with hypertension, etc.). The global knowledge repository may and typically does contain a large amount of information irrelevant to the task at hand, but has a structure which permits extraction of potentially relevant assertions based on the application of biological criteria specified by a user.

Nodes may be, by way of non-limiting examples, biological molecules including proteins, small molecules, ions, genes, ESTs, RNA, DNA, transcription factors, metabolites, ligands, trans-membrane proteins, transport molecules, sequestering molecules, regulatory molecules, hormones, cytokines, chemokines, histones, antibodies, structural molecules, metabolites, vitamins, toxins, nutrients, minerals, agonists, antagonists, ligands, or receptors. The nodes may be drug substances, drug candidate compounds, antisense molecules, RNA, RNAi, shRNA, dsRNA, or chemogenomic or chemoproteomic probes. Viewed from a chemistry perspective, the nodes may be protons, gas molecules, small organic molecules, amino acids, peptides, protein domains, proteins, glycoproteins, nucleotides, oligonucleotides, polysaccharides, lipids or glycolipids. Proceeding to higher order models, the nodes may be protein complexes, protein-nucleotide complexes such as ribosomes, cell compartments, organelles, or membranes. From a structural perspective, they may be various nanostructures such as filaments, intracellular lipid bilayers, cell membranes, lipid rafts, cell adhesion molecules, tissue barriers and semipermeable membranes, collagen structures, mineralized structures, or connective tissues. At still higher orders, the nodes are cells, tissues, organs or other anatomical structures. For example, a model of the immune system might include immunoglobulins, cytokines, various leucocytes, bone marrow, thymus, lymph nodes, and spleen. In simulating clinical trials the nodes may be, for example, individuals, their clinical prognosis or presenting symptoms, drugs, drug dosage levels, and clinical end points. In simulating epidemiology, the nodes may be, for example, individuals, their symptoms, physiological or health characteristics, their exposure to environmental factors, substances they ingest, and disease diagnoses. Nodes may also be ions, physiological processes, diseases, disease processes, translocations, reactions, molecular complexes, cellular components, cells, anatomical parts, tissues, cell lines, and protein domains.

Descriptors may represent biological relationships between nodes and include, but are not limited to, non-covalent binding, adherence, covalent modification, multi-molecular interactions (complexes), cleavage of a covalent bond, conversion, transport, change in state, catalysis, activation, stimulation, agonism, antagonism, up regulation, repression, inhibition, down regulation, expression, post-transcriptional modification, post-translational modification, internalization, degradation, control, regulation, chemo-attraction, phosphorylation, acetylation, dephosphorylation, deacetylation, transportation, and transformation.

A preferred form of descriptors for use in the invention are case frames extracted from the representation structure which permit instantiation and generalization of the models to a variety of different life science systems or other systems. Case frames are described in detail in co-pending, co-owned U.S. patent application Ser. No. 10/644,582, the disclosure of which is incorporated by reference herein. Descriptors may comprise quantitative functions such as differential equations representing possible quantitative relationships between pairs of nodes which may be used to refine the network further. Descriptors may also comprise qualitative features that either cannot be measured or described easily in an analytical or quantitative manner, or because of insufficient knowledge of a system in general or the feature itself, it is impossible to be described otherwise.

The knowledge assembly process may be conducted on disparate systems and the output combined into a consolidated assembly which constitutes a model. Furthermore, a knowledge assembly constructed on disparate systems can be accessed as a cohesive model by accessing the fragments of the model in a distributed fashion. A model represents a hypothesis explaining the operation of systems, i.e., capable of producing, upon simulation, predicted data that matches the actual data that serves as the fitness criteria. The hypothesis can be tested with further experiments, combined with other models or networks, refined, verified, reproduced, modified, perfected, corrected, or expanded with new nodes and new assertions based on manual or computer aided analysis of new data, and used productively as a biological knowledge base. Models of portions of a physiological pathway, or sub-networks in a cell compartment, cell, organism, population, or ecology may be combined into a consolidated model by connecting one or more nodes in one model to one or more nodes in another.

Each fact within a knowledge base or assembly is referred to herein as an assertion. Assertions represent facts relating existing objects in a system, or a fact about one object in the system and some literal value, or any combination thereof. In various embodiments, assertions may represent knowledge such as RNA, proteomic, metabolite, or clinical knowledge from sources such as scientific publications, patient data, clinical trial data, compliance data, chemical data, medical data, hypothesized data, or data from biological databases.

Construction of an assembly begins when an individual specifies, via input to an interface device, biological criteria designed to retrieve from the knowledge repository all assertions considered potentially relevant to the issue being addressed. Exemplary classes of criteria applied to the repository to create the raw assembly include, but are not limited to, attributions, specific networks (e.g., transcriptional control, metabolic), and biological contexts (e.g., species, tissue, developmental stage). Additional exemplary classes of criteria include, but are not limited to, assertions based on a relationship descriptor, assertions based on text regular expression matching, assertions calculated based on forward chaining algorithms, assertions calculated based on homology, and any combinations of these criteria. Key words or word roots are often used, but other criteria also are valuable. For example, one can select assertions based on various structure-related algorithms, such as by using forward or reverse chaining algorithms (e.g., extract all assertions linked three or fewer steps downstream from all serine kinases in mast cells). Various logic operations can be applied to any of the selection criteria, such as “or,” “and,” and “not,” in order to specify more complex selections. It is the diversity of sets of criteria that can be devised, and the depth of the assertions in the global knowledge base that enable the flexibility inherent in the invention.

Assertions selected in accordance with the invention in the form of data entries that satisfy a set of specified criteria are retrieved from the knowledge base and then reassembled into a sub-knowledge base or assembly comprising a subset of interrelated nodes and descriptors potentially relevant to the system under study. This subsetting creates a new biological model. This model typically comprises far fewer assertions than the global knowledge base, and serves as a starting point on the path to producing a more useful and focused assembly. It is then transformed or refined by automatic routines in the software application that created it and by application of tools by the individual conducting the exercise. It can be augmented and integrated with other information, including, but not limited to, assertions derived from the literature by a curator who considered them to be relevant to the biological system.

Assemblies created by the present invention usually are better than the global knowledge base or repository they were derived from in that they typically are more predictive and descriptive of real biology. This achievement of the invention rests on the application of logic during or after compilation of the raw data set so as to augment the initially retrieved data, and to improve and rationalize the resulting structure as noted herein. This can be done automatically during construction of the assembly, for example, by programs embedded in computer software, or by using software tools selected and controlled by the individual conducting the exercise.

An assembly in many ways is structurally identical to a global knowledge repository, but is smaller and much more focused on the topic or problem under consideration, more tractable computationally, and isolated either physically or virtually so as to be customized for a particular project, and to facilitate compliance with restrictive use or disclosure obligations that may be imposed by a data source. Additionally, an assembly often will have the characteristics of a work in progress, being altered and improved, probed and corrected over the course of the exercise. An assembly can be stored in a computable format at any time, or at every iteration, and added back to the global knowledge base.

The production of a valuable assembly thus involves a subsetting or segmentation process applied to a global repository, followed by data transformations or manipulations to improve, refine and/or augment the first generated assembly so as to perfect the assembly and adapt the assembly for analysis. This is accomplished by implementing a process such as applying logic to the resulting database to harmonize it with real biology. For example, the criteria can ask for all proteins expressed in human myocyte and the repository may include mouse myocyte proteins some of which are not present in human tissue, so these data are removed from an assembly probing myocyte physiology in humans. An assembly may be augmented by insertion of new nodes and relationship descriptors derived from the knowledge base and based on the assumptions set forth above (and many other logical assumptions that are possible). An assembly may be filtered by excluding subsets of data based on other biological criteria. The granularity of the system may be increased or decreased as suits the analysis at hand (which is critical to the ability to make valid extrapolations between species or generalizations within a species as data sets differ in their granularity). An assembly may be made more compact and relevant by summarizing detailed knowledge into more conclusory assertions better suited for examination by data analysis algorithms, or better suited for use with generic analysis tools, such as cluster analysis tools.

An assembly may be updated periodically as knowledge advances, and the respective evolving assemblies can be saved to show the progression of knowledge in the area. An assembly may be augmented in various ways, including having a curator add new data from a structured or unstructured database or add data derived from literature. An assembly also may be incorporated back into a global repository so that new assertions may be used as raw material for creation of a different assembly.

The underlying knowledge representation of a knowledge repository is designed to capture knowledge with considerable detail and without bias as to the use of the knowledge. Reasoning with a network of this complexity can be difficult. Therefore, methods and systems of the invention embody a flexible framework for manipulating the knowledge in stages, creating derivative assemblies by the application of well-defined rules or procedures. These derivative assemblies are constructed to enable subsequent rounds of reasoning on the assemblies.

Assemblies may be used to model any biological system, no matter how defined, at any level of detail, limited only by the state of knowledge in the particular area of interest, access to data, and (for new data) the time it takes to curate and import it. In one embodiment, assemblies may be used to update models continuously or intermittently as new relevant data becomes available so as to record and provide a vehicle to better understand biology. In another embodiment, assemblies may be used to display biological systems in whole or in part in various formats for human visual inspection and analysis.

Assemblies may also be used to query biological systems in various ways to mine new biological knowledge (e.g., overlay different assemblies to discern differences). In various embodiments, assemblies may be used to: (a) predict physiological behavior (e.g., drug efficacy and toxicity) in humans from analysis of experiments conducted on animals; (b) to find ideal biomarkers (substances in body fluids easily detected or quantitated to provide predictions informative of the presence of disease, its prognosis, whether the patient will respond to drug X, disease severity, etc.); or (c) to learn how to segment members of a population so as to improve outcomes and avoid adverse events in clinical trials.

Assemblies may further be used to study biology by comparing different assemblies (e.g., human to mouse, diseased tissue to healthy tissue, adipose physiology under various different dietary constraints). Assemblies may be used to compare the biology of tissues at different time points during disease development, progression or healing, or to determine the effect of various perturbations within any desired biological system, such as drug effects, or the effect of some other environmental influence. Assemblies may be used to map data (i.e., to show the effect on a biological system of perturbations to one or more components of the system based on import of experimental data). In further embodiments, assemblies may be used to implement logical simulations, to evaluate data sets not present in a global repository at the time of the original assembly construction (e.g., to retest a hypothesis based on new experimental data), to hypothesize pathways and discern complex and subtle cause and effect relationships within a biological system, and to discern disease etiology, understand toxic biochemical mechanisms, and predict toxic response.

New knowledge may be discovered by using the assemblies, for example, with epistemic engines. Epistemic engines are described in detail in co-pending, co-owned U.S. patent application Ser. No. 10/717,224, the disclosure of which is incorporated by reference herein. Epistemic engines are programmed computers that accept biological data from real or thought experiments probing a biological system, and use them to produce a network model of protein interactions, gene interactions and gene-protein interactions consistent with the data and prior knowledge about the system, and thereby deconstruct biological reality and propose testable explanations (models) of the operation of natural systems. The engines identify new interrelationships among biological structures, for example, among biomolecules constituting the substance of life. These new relationships alone or collectively explain system behavior. For example, they can explain the observed effect of system perturbation, identify factors maintaining homeostasis, explain the operation and side effects of drugs, rationalize epidemiological and clinical data, expose reasons for species success, reveal embryological processes, and discern the mechanisms of disease. The programs reveal patterns in complex data sets too subtle for detection with the unaided human mind. The output of the epistemic engine permits one to better understand the system under study, to propose hypotheses, to integrate the system under study with other systems, to build more complex and lucid models, and to propose new experiments to test the validity of hypotheses.

The functionality of the systems and methods disclosed herein may be implemented as software on a general purpose computer. In some embodiments, a computer program may be written in any one of a number of high-level languages, such as FORTRAN, PASCAL, C, C++, LISP, JAVA, or BASIC. Further, a computer program may be written in a script, macro, or functionality embedded in commercially available software, such as EXCEL or VISUAL BASIC. Additionally, software could be implemented in an assembly language directed to a microprocessor resident on a computer. For example, software could be implemented in Intel 80×86 assembly language if it were configured to run on an IBM PC or PC clone. Software may be embedded on an article of manufacture including, but not limited to, a storage medium or computer-readable medium such as a floppy disk, a hard disk, an optical disk, a magnetic tape, a PROM, an EPROM, or CD-ROM.

Assembly Construction

The invention allows creation of knowledge assemblies by extracting from a global repository and then adding new knowledge through curation and other methods. In one example, new knowledge is added to a global repository in a stepped, application-focused process. First, general knowledge not already in the global repository (e.g., additional knowledge regarding cancer) is added to the global repository. Second, base knowledge is gathered in the field of inquiry for the intended application (e.g., prostate cancer) from the literature, including, but not limited to, text books, scientific papers, and review articles. Third, the particular focus of the project (e.g., androgen independence in prostate cancer) is used to select still more specific sources of information. This is followed by using experimental data to guide the next step of curation and knowledge gathering. For example, experimental data may show which genes and proteins are involved in the area of focus. By curating the literature relating to genes and proteins in the data, a sub-assembly can be created that is focused on the area of interest.

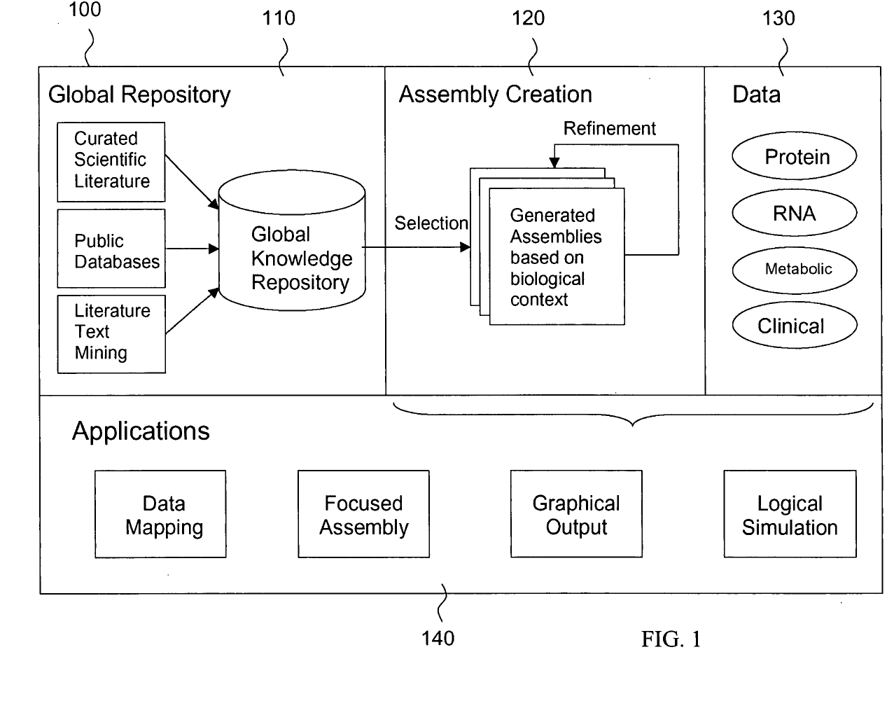

An illustrative overview of a system in accordance with the invention is shown in FIG. 1. In this diagram, the system 100 is used for discovering new biological knowledge. In phase 110, a global knowledge repository is created by inputting information (e.g., curated scientific data from the literature, public databases, and information from literature text mining) into a computer database. In phase 120, a subset of the information in the global knowledge repository is extracted to generate knowledge assemblies based on biological content. The knowledge assemblies are then refined. In phase 130, experimental data (e.g., data relating to proteins, RNA, metabolic activity, clinical information, etc.) is used to guide curation and knowledge gathering. In phase 140, knowledge assemblies can be used in various applications including, for example, data mapping, focused assembly by application of pathfinding, graphical output and logical simulation.

Algorithms may be used to create derivative assemblies. In some embodiments, algorithms may be expressed as a computer program and may be used to create derivative assemblies as data objects within a programming framework. An exemplary algorithm performs one or more transformations on the existing assemblies to generate a new assembly. Transformations can be accomplished, for example, by any of the following techniques: (a) selecting assertions from existing assemblies and inserting the selected assertions into a new assembly under construction; (b) summarizing nodes and assertions from existing assemblies and inserting the summarized nodes and assertions into an assembly; (c) applying mathematical set theory operations on the nodes and assertions of existing assemblies and inserting the nodes and assertions resulting from those operations into an assembly; (d) applying assembly operations to existing assemblies to create an assembly that will be used for further transformations; or (e) applying a combination of any of the techniques listed above.

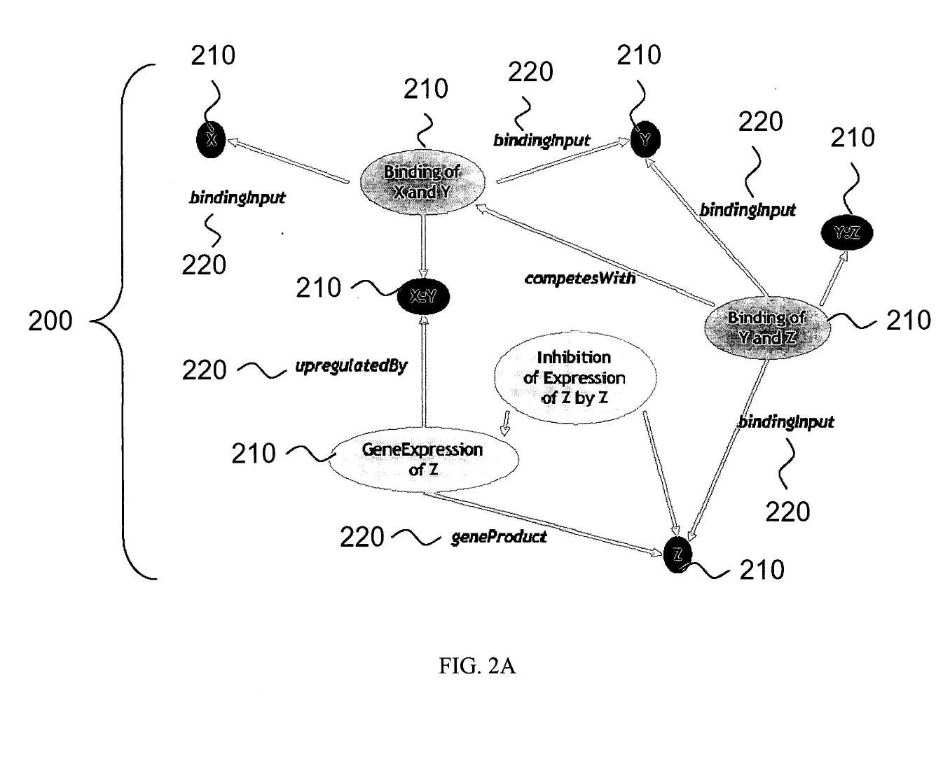

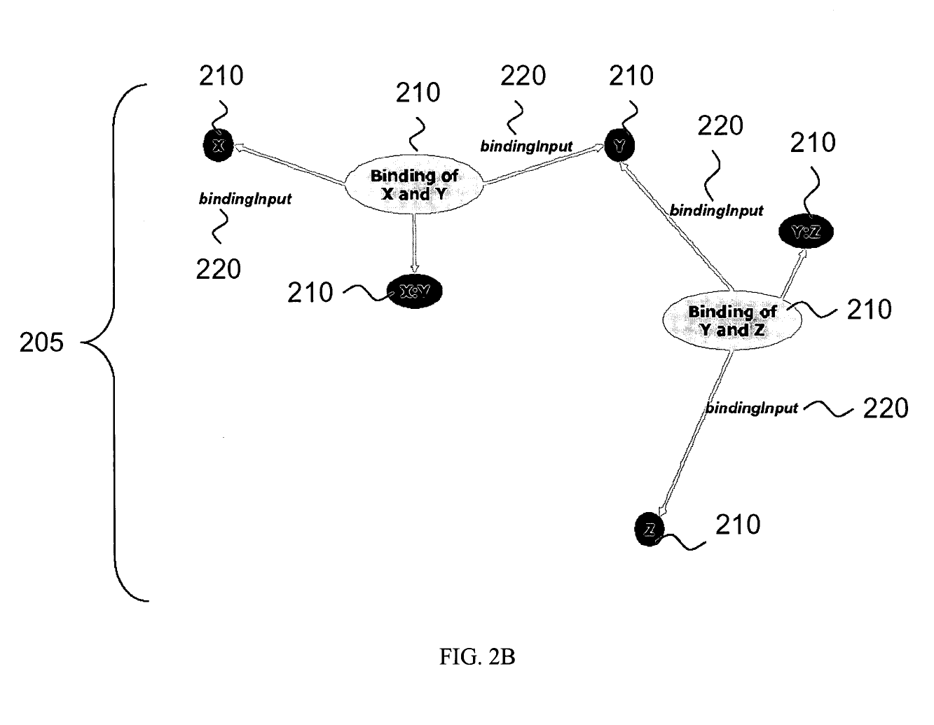

The simplest form of a transformation of an assembly is to create a subset of the assembly. For example, a subset of an assembly may contain a subset of the nodes and descriptors in the original assembly. A subset is essentially the result of a query which selects nodes and assertions based on a set of criteria. Those criteria may be procedurally defined, i.e. the selection may be the result of some algorithm which iteratively or recursively explores nodes and descriptors which embody the assembly. For example, as shown in FIG. 2A, an original network 200 of nodes 210 and descriptors 220 was transformed, as shown in FIG. 2B, to create a subset network 205 of nodes 210 descriptors 220 only of the type “A bindingInput B” and therefore excluding all others. “A bindingInput B” is an assertion that relates a class of molecular binding processes A to a class of molecular entities B (i.e., molecule or complex).

In some embodiments, an assembly may take the form of one or more database tables, each having columns and rows. In these embodiments, the transforming or subsetting of a global knowledge base to an assembly can be accomplished, for example, by selecting rows representing assertions from a database table that match a user's selection criteria. It should be understood that a knowledge base or assembly in the form of a database is only one way in which information may be represented in a computer. Information could instead be represented as a vector, a multi-dimensional array, a linked data structure, or many other suitable data structures or representations.

One aspect of an assertion is its attribution. An attribution represents the source of the assertion, such as a scientific article, an abstract (e.g., Medline or PubMed), a book chapter, conference proceedings, a personal communication, or an internal memorandum. An assembly can be created by selecting descriptors whose attribution meet some specification, such as a match by type of the attribution source, name of the attribution source, or date of the attribution source. For example, one might select all assertions whose attribution is a node representing a journal article published in the year 2001 or later.

Another aspect of an assertion is its biological context. Assertions associated with a specific biological context may be selected. Biological context refers to, for example, species, tissue, body part, cell line, tumor, disease, sample, virus, organism, developmental stage, or any combination of the above. A further aspect of an assertion is its trust score, a measure of the level of confidence that the assertion reflects truly representative, real biology and is reproducible. Assertions can also be selected on the basis of a trust score. A minimum threshold is set and any assertions meeting or exceeding the threshold are selected.

Subsets of a knowledge base can also be made using specifications that define a complex pattern of assertions between nodes. All the sets of nodes and assertions which meet the criteria of the pattern embody the subset. In one embodiment, a search algorithm can filter the knowledge base to generate a list of biological entities that satisfy the stated pattern. For example, a structure search can be used to generate the subset of all reactions that have a product which is phosphorylated and whose catalyst is a molecular complex. This search will find all phosphorylation reactions that are catalyzed by a molecular complex, while avoiding phosphorylation reactions that are catalyzed by a single protein.

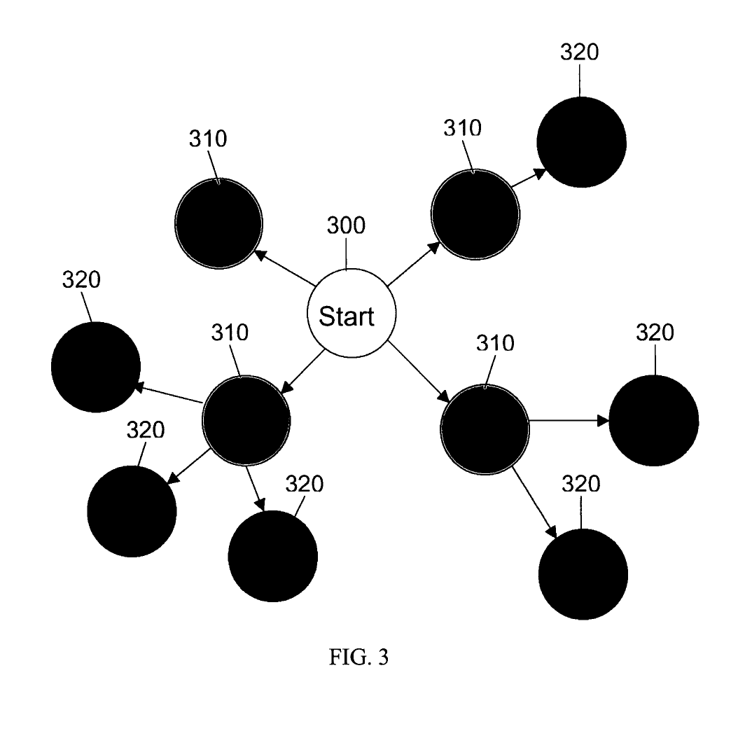

In another embodiment, subsets can be generated using pathfinding algorithms including radial, shortest path, and all paths pathfinding. Radial pathfinding is useful to discover how one biological entity is functionally or structurally connected to another biological entity. For example, if a given cell contains a mutant form of P53, one may want to discover its effect on molecules upstream or downstream from the mutant gene product. An algorithm for discovering this information can start from a particular node and find all nodes that are connected to the node for a predetermined number of steps removed from the node. If directionality is important (e.g., as in reactions), the algorithm can be instructed to follow links only in the direction indicated by the pathfinding criteria. Radial pathfinding can be applied in several steps. For example, a two-step radial pathfinding search will involve starting from a node, finding its immediate connected nodes, and then finding the immediate connected nodes of those nodes. This process can be applied to as many steps as needed. This analysis may be used to determine and predict the expected changes of perturbing a given node. This analysis may be displayed to the user to elucidate how a change might propagate through the knowledge base, and thereby to discover its real effect on a biological system. FIG. 3 shows an example of the progression of a two-step radial pathfinding search starting from a specified start node 300. In the first step of the search, connected nodes 310 are found. In the second step of the search, connected nodes 320 are found. The result of this radial pathfinding search is the combination of all nodes and assertions as shown in the FIG. 3. A pathfinding search optionally can be configured to follow only specific descriptors, to ignore certain nodes that may be ubiquitous or uninformative, or to stop finding new nodes when certain nodes are encountered.

In large biological networks, there usually are multiple paths between any two entities. Often times, the shortest path is the most useful for analysis. An algorithm for determining the shortest path in a network starts by performing a breadth-first radial pathfinding from each of the two given starting nodes. Once a common node is found, the path is published as the shortest path between the nodes. In order to determine the pathways among several nodes, the shortest path algorithm discussed above can be run until all pathways among the nodes are found. In this technique, one starts a radial pathfinding search from every one of the start nodes. Then, the paths being followed are recorded in every radial search. The union of all paths from the start nodes to the target nodes is the result of this algorithm. As this approach tends to increase exponentially in the number of pathways and nodes, the algorithm may be limited to follow a pre-designated number of steps. For example, a three-step search will only generate all pathways that exist between the given origin nodes by doing a three-step radial search out from each node. The results of this pathway algorithm can be displayed, for example as a sorted list of pathways starting from the shortest or largest, or as a merged graph.

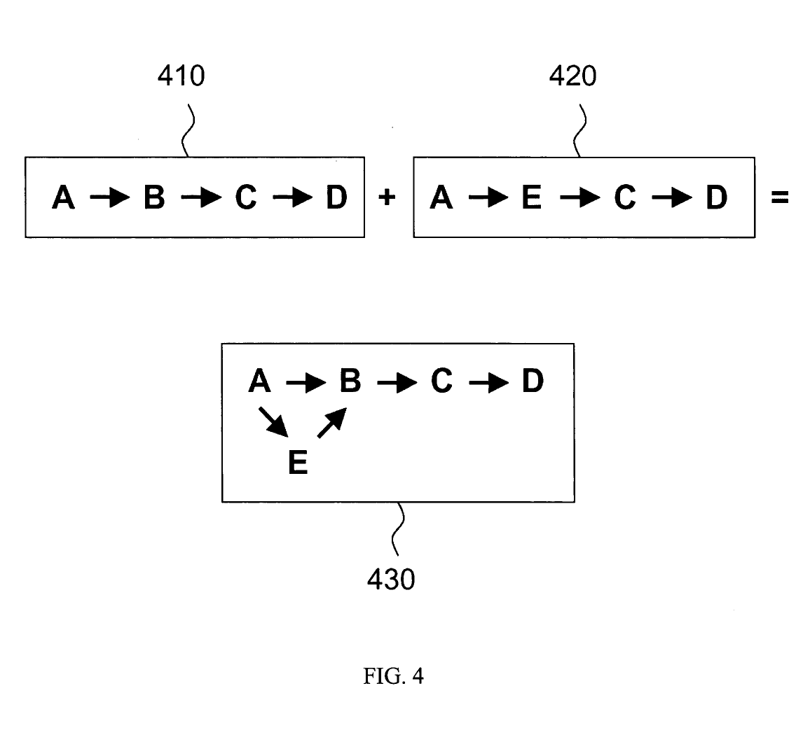

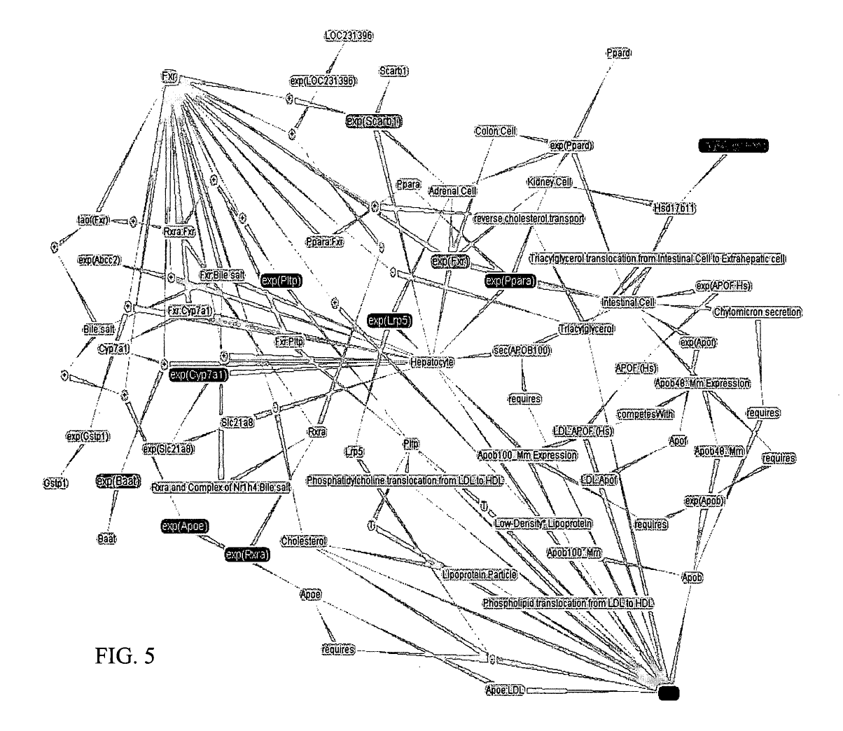

A merged graph is generated by merging together all of the pathways traversed up to a specific length in the case of a radial search or by merging the set of pathways that link any of the source nodes to any of the target nodes. This is accomplished by merging two pathways at a time, until only a single graph containing all nodes and assertions emerges. An example of merging two pathways involves taking all common nodes and assertions and merging them into combined pathway as shown in FIG. 4. In this diagram, since nodes A, B, and D are shared between pathway 410 and pathway 420, these nodes are represented only once in the combined pathway 430. Node B occurs in pathway 410 and node E occurs in pathway 420, and they are also represented in the combined pathway 430. FIG. 5 shows the result of merging all pathways into a single graph based on a radial pathway search between a start node “FXR” (in the upper left-hand corner of the diagram) and a target node “LDL” (in the lower right-hand corner of the diagram). This type of analysis permits study of the implications of observed changes in gene expression studies or changes in concentrations of proteins and metabolites. The analysis is used to show how the changed entities relate to one another so one can discern the dependent changes and find changes that are central to the experiment at hand.

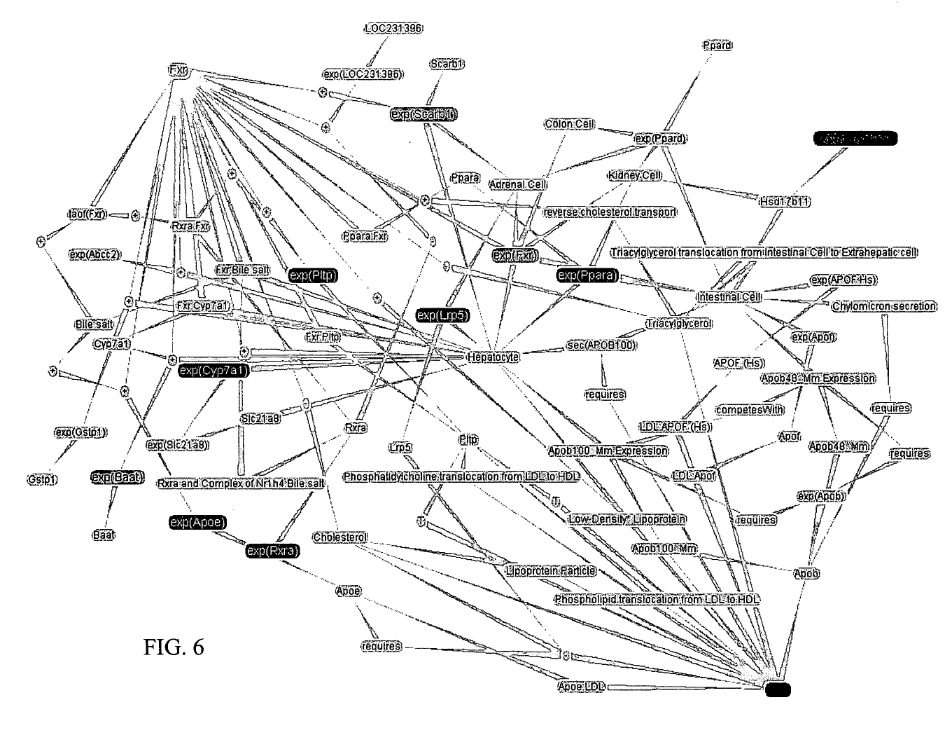

The matrix method is another way of studying the changes in a knowledge assembly graph. Given a list of nodes of interest (e.g., statistically significant, highly modulated RNA in an experiment) the nodes are placed in a matrix with each node placed as an entry in a column and a row. The shortest path is then generated for every pair of nodes (redundant pairings are ignored). All the generated pathways are then merged as explained above. The matrix method can also be applied by not only finding one path for each cell in the matrix, but by generating multiple pathways. This can be done in several ways: (1) generating all pathways for each pair; (2) generating the top “n” pathways starting with the shortest or longest; and (3) generating all the top “n” pathways that are no more than some pre-determined number of steps long. The matrix method also is useful in determining how a set of biological entities are related to one another. FIG. 6 shows the result of a matrix method analysis among three nodes, “Acox1”, “LDL” and “FXR” after merging all of the shortest paths between each pair of nodes.

A derived network is not limited to operations which subset, simplify or summarize the starting network. The derivation may embody a theory about the knowledge, one which allows the inference of new facts based on other facts. A primary example of this is the theory that biological mechanisms are conserved and that mechanism is dependent on gene and protein sequence. Thus, if a mechanism is known in one species, that mechanism may be inferred to exist in another species if all the genes/proteins involved in the mechanism have highly similar—homologous—counterparts in the second species. This technique is used to augment knowledge assemblies which are focused on a single type of organism. For example, an assembly focused on human biology can be augmented by considering facts about mouse biology, determining which “mouse” facts meet the criteria for homology to human, and then creating the homologous human facts in the assembly. The degree of homology is determined by homology scores, computed by comparing the sequences of the genes or proteins. These scores allow thresholds of similarity to be set for a given purpose—in some embodiments the criteria for homology may be set loosely, allowing importation of facts from the context of other organisms. In other embodiments, the threshold may be set high, admitting only mechanisms based on the most similar genes and proteins.

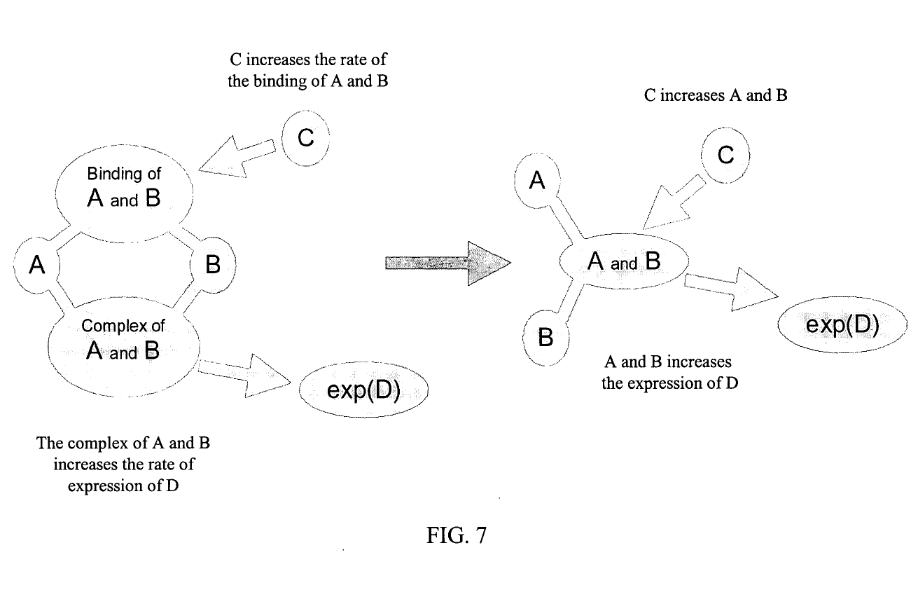

A straightforward example of a derived network is one formed by collapsing nodes which do not need to be distinguished as separate concepts. For example, the representation distinguishes the act of a “binding”—a process where entities form a complex—from a “complex”—the result of a binding event. This distinction is distracting in many contexts—especially when visualizing a network in a graph, or when grouping proteins by their binding interactions. FIG. 7 shows an example of a network transformed by collapsing nodes. In this diagram, the binding of A and B is merged with the node representing the complex of A and B and the new node is substituted in place of either of the original nodes in all cases.

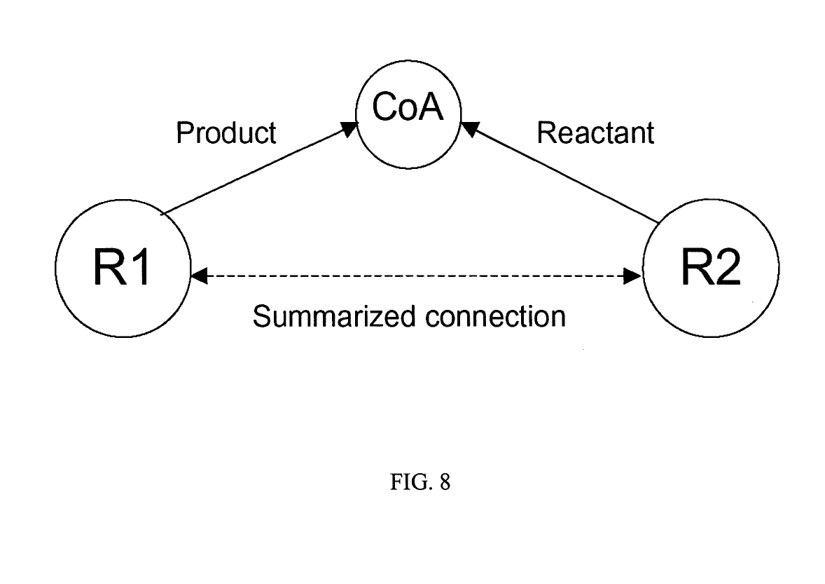

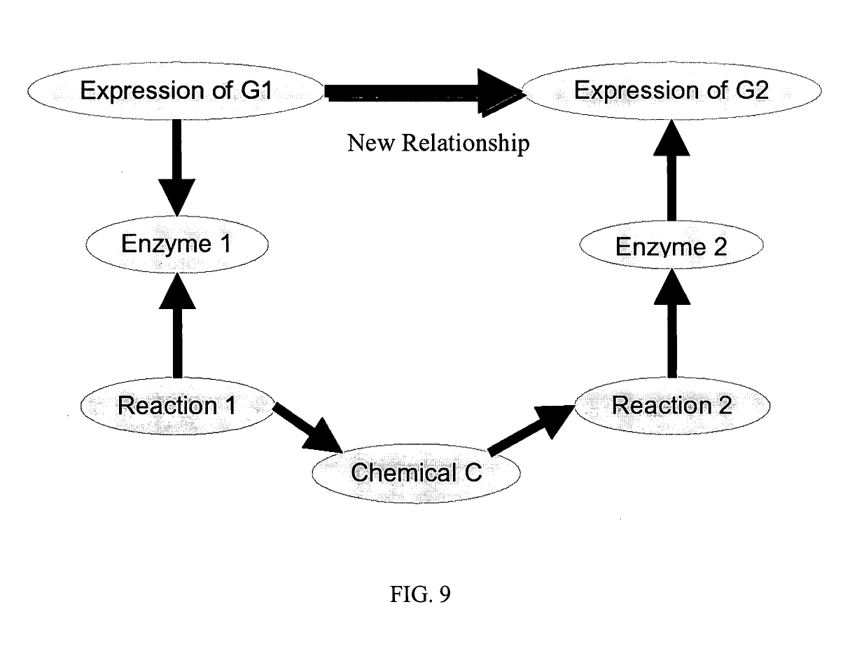

An assembly may be transformed by a summarization process. A summarization begins with a subsetting process where sets of nodes matching a specification are selected. Each of those sets may be replaced by some new set of nodes and assertions, typically a simpler pattern such as a single assertion between two nodes. FIG. 8 shows an example of summarization of two reactions represented as R1 and R2 that share a common metabolite CoA. The assertions in this example are “R1 reactant M” and “R2 product M.” The summarized connection between reactions R1 and R2 is represented as the assertion “R1 newRelationship R2.” A more complex derivation may be used to create a network of simple links, substituting a simple link in place of a complex pattern of relationships between two nodes. This can be viewed as a “summarization” process. In this example, a relationship is created between genes when they meet the following criteria: (1) each has a gene product which acts as an enzyme in a reaction; and (2) a reaction catalyzed by one gene product creates a product which is in turn a reactant in a different reaction catalyzed by the other gene product. The resulting derived network, as shown in FIG. 9, links the genes G1 and G2 which are adjacent in a derived assembly. This derived assembly has many applications. For example, if it is annotated with gene expression data, an algorithm may then find groups of co-regulated genes which are near each other in the derived assembly. This corresponds to finding reaction pathways which are commonly regulated.

Transformations to the assembly may be performed by mathematical set theory operations. These operations include, for example, intersection, difference and union. Set operations can be used to compare assemblies. All set operations assume that there are two existing assemblies. Using the intersection operation, each assertion in a first assembly, the same assertion is checked to see if it appears in a second assembly. If it does appear in the second assembly, the assertion is added to an intersection assembly. Nodes that are mentioned in any assertion in the intersection assembly are also selected from the first assembly and added to the intersection assembly. Using a difference operation, for each assertion in a first assembly, the same assertion is checked to see if it does not appear in a second assembly. If it does not appear in the second assembly, the assertion is added to a difference assembly. Nodes that are mentioned in any assertion within the difference assembly are also selected from the first or second assemblies and added to the difference assembly. Using a union operation, a union assembly is created. All assertions in a first assembly are added to the union assembly. For each assertion in a second assembly, if it does not exist in the union assembly, the assertion is then added to the union assembly. Nodes that are mentioned in the union assembly are also selected from the first or second assemblies. The union operation is another way of stating that two or more assemblies may be merged.

An example of a comparison technique in accordance with the invention is measuring the progression of a knowledge assembly over time. This can be accomplished by taking a sequence of assemblies that are created over time, determining the difference between each pair in the sequence. Additionally, two or more assemblies may be compared in accordance with the invention. For example, using an intersection of two assemblies, where the two assemblies are not identical, the intersection of assertions in the two assemblies is determined. The intersection contains the assertions that appear in both assemblies. Using the difference of two assemblies, where the two assemblies are not identical, the difference of assertions in the two assemblies is determined. The difference contains the assertions that appear in one assembly but not the other. Comparisons between assemblies can be useful in explaining similarities and differences between biological systems. For example, one assembly could represent a normal system and another assembly could represent a diseased system. It would be informative to a scientist to determine the similarities and differences between to the two systems.

Assembly Mining Tools

The present invention may include analyzing an assembly to discover new biological knowledge. Analyzing includes, but is not limited to, algorithmic analysis, which can be performed by computers or individuals. Algorithms that incorporate pathfinding, homological reasoning or simulation-based reasoning can derive new assertions that may be added back to augment the assembly. Assemblies can also be refined and augmented by homology transformation, relying on the assumptions that (1) the physics and fundamental biochemical properties and interactions of matter remain constant under typical biological conditions, and (2) homologous structures have identical or analogous function. For example, if a global knowledge base includes data that when molecule A collides with molecule B in a nerve cell that complex C is produced, it can be assumed that A+B=C also holds when A and B collide in a liver cell. If the liver model assertions of the global knowledge base includes node A and node B, but not the descriptor stating that they together form complex C, the latter information can be imported into a liver assembly during its compilation. Whole cascades of biological activity can be imported into an assembly using such logic. Similarly, if a global knowledge base contains the information that a mouse protein M binds to mouse receptor R to initiate renal tubule repair in mouse, and human biology assertions in the knowledge base include a node homologous to mouse protein M and another homologous to receptor R, then the interaction and potential downstream events may be imported from the mouse to an assembly directed toward a human biological system. Furthermore, an assembly may be combined with another, generated using different criteria, and then the logical inconsistencies and redundancy removed to produce an even better, more complete, or more focused biological model.

Graphical Output Techniques

A knowledge assembly can be displayed visually as a graph of nodes connected by connections representing biological relationships between and among nodes. These graphs can be inspected by a scientist to understand the biological system and to facilitate the discovery of new biological knowledge about life sciences-related systems. Using assemblies to discern biologically relevant insights into how a system behaves can be extremely valuable in drug research and development, and for developing a variety of therapies. The techniques described herein can be used to develop biologically relevant insights using assemblies created by methods and systems of the invention. Visualization techniques can also be used to display knowledge and associated data to enhance user understanding and recognition of relationships among entities that may emerge as patterns and clusters.

Having generated graphs using any of the above techniques, one may want to get a better idea of the biological context of the pathways. This can be done by starting from every node in the input graph and doing a n-step radial search out from each node. This step “expands” the nodes and the size of the graph. By color coding the nodes to indicate modulation (as determined by experimental data), one is able to discern changes of interest that are functionally or structurally proximal the original graph of interest, in other words, the biological context.

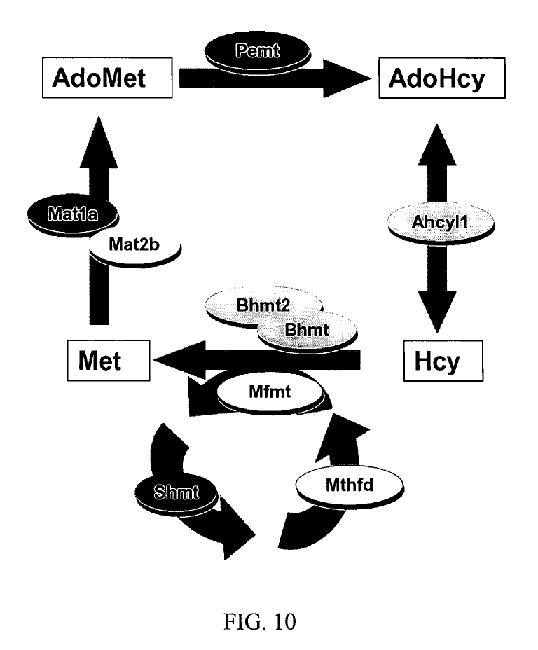

Experimental data may be mapped onto an assembly by matching measurements from experiments to the assertions in the assembly which represent the quantities measured. Mapping, in this context, means superimposing visually recognizable indicia, such as color, onto a pathway map so as to indicate which nodes are involved in a process. For example, this may be done by matching nodes that represent gene expression processes to the levels of mRNA measured by microarrays or by other techniques such as RT-PCR. Nodes representing abundance of proteins may be matched to data from proteometric measurements. Nodes representing abundance of chemicals may be matched to data from metabolomic measurements. Once mapped, the data can be processed to create simpler qualitative attributes of the node that facilitate display or analysis algorithms. For example, fold change data may be summarized based on user-controlled thresholds, annotating nodes with additional qualitative attributes such as “up” or “down,” allowing the use of straightforward display or analysis algorithms. Fold change data may also be shown by shading, as shown for example in FIG. 10, where the shading of each expressed gene in the diagram (e.g., Mat1a, Mat2b, Pemt, Ahcyl1, Bhmt, Bhmt2, Mfmt, Shmt, and Mthdf) is indicative of its fold change in an experiment (i.e., the darker the shading, the greater the fold change).

Logical simulation may also be utilized in accordance with the invention. Logical simulation refers to a class of operations conducted on an assembly wherein observed or hypothetical changes are applied to one or more nodes in the assembly and the implications of those changes are propagated through the network based on the causal relationships expressed as assertions in the assembly. A logical simulation can either be forward, where the effects of changes are inferred and are propagated downstream from the initial points of change, or it can be backward where the possible causes are inferred and are propagated upstream from the initial points of change. In either case, one result of a logical simulation is a new, derived network, comprised of the nodes and assertions that were involved in the propagation of cause or effect. This derived network embodies a hypothesis about the system being studied.

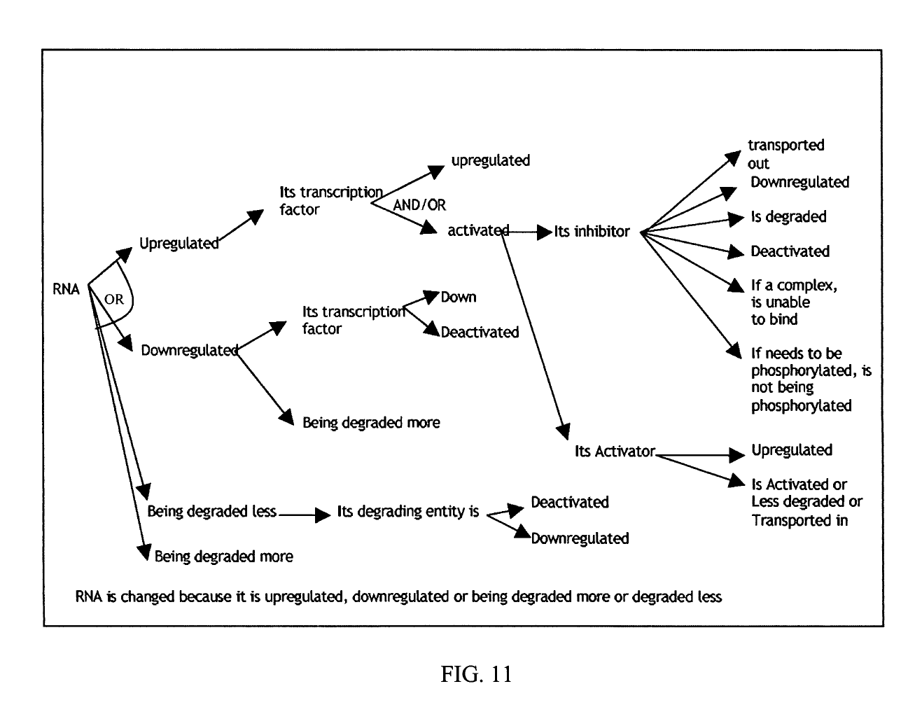

For example, in the case of a backward simulation based on observed changes in RNA expression levels, FIG. 11 shows paths of inference to find upstream causes starting with an observed change in mRNA levels for a particular gene. One specific chain of causation could be as follows: a phosphorylation of a transcription factor by a kinase such that the kinase changes the activity of the transcription factor can in turn induce changes in the expression of genes controlled by that transcription factor. This diagram provides a “pseudo code” description of the inferences that are then performed to find possible causes of each of the observed RNA changes. The types of assertions to be explored are not limited to those in this diagram. Any assertion in the assembly that represents a causal biological linkage may be included in this type of analysis. In turn, each of the possible causes may then be explored to find their respective possible causes. The process may be repeated for as many steps as desired, annotating nodes in the assembly according to their possible role in the causation of the observed changes.

The resulting derived network embodies a hypothesis about the possible causes of the observed data. Moreover, depending on the methods of propagation of causality, it may further be considered a hypothesis about the most implicated and most consistent possible causes of the observed data, i.e. a set of possible causes ranked by objective criteria. This technique is not limited to RNA expression data, but rather may work with any set of changes that can be expressed in the representation system, including but not limited to proteometric data, metabolomic data, post-translational modification data, or even reaction rate data.

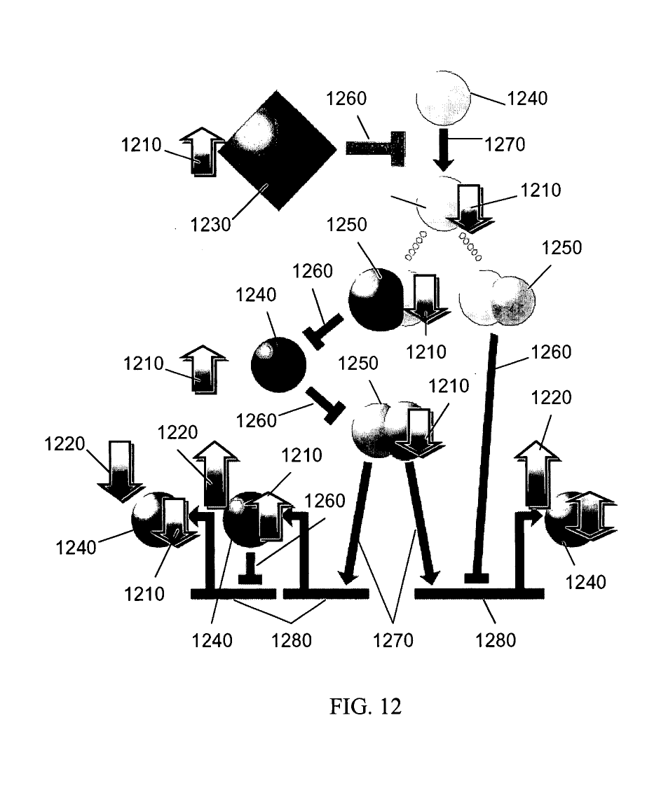

FIG. 12 is a manually composed diagram which shows propagation of predicted changes 1210 in a forward simulation being compared with observed expression changes 1220. This diagram illustrates the propagation of predicted protein changes 1210 based on an increase in the amount of a compound 1230 through a known pathway. In this diagram, spheres 1240 represent proteins. Pairs of adjacent spheres 1250 indicate complexes of proteins. Thin arrows with T-shaped heads 1260 indicate inhibitions or causal decreases. Thin arrows with pointed heads 1270 indicate an activation or causal increase. Gene expression relationships are indicated by the arrows 1280. The diagram is intended to clarify the way in which changes predicted by a hypothesis may be compared with observed data.

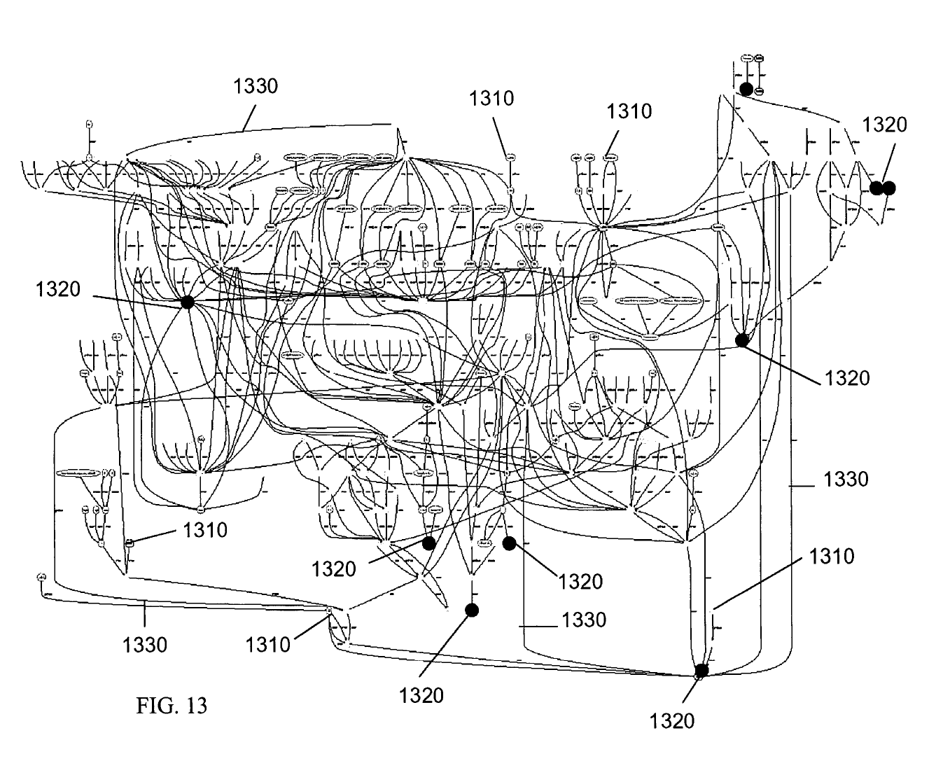

FIG. 13 is a diagram generated by backward simulation from nine observed expression data points 1320, followed by pruning of the graph to show only the connections 1330 which support the primary hypotheses. Each node 1310 in this figure represents either a gene, protein, or compound. Nine of these nodes 1320 represent changes in expression of genes in response to dietary polyunsaturated fatty acids. The rest of the diagram is generated by exploring the assembly to find possible nodes 1310, which if changed, could explain one or more of the observed nine changes 1320 and then removing nodes 1310 and connections 1330 such that only the best explanations are shown.

Derived networks may be created as data objects within a general-purpose programming framework, such as a scripting language. These data objects may be saved, loaded, and acted on by specific operators, such as the pathfinding or logical simulation procedures described above. In addition, the data objects may be operated on by the standard functions of the programming framework. Because both the input and the output of these operations include the derived networks, multiple steps of processing may be combined in larger procedures, procedures which embody biologically significant inferences, procedures which embody theories and techniques of automatically processing biological datasets and knowledge. Multiple derived networks may be created by different criteria and then compared, merged and otherwise operated on. Multiple hypotheses, as embodied in these networks, may be evaluated, compared, and ranked.

One example of a method comprised of techniques herein above would be as follows: (1) load a set of expression fold-change data to the assembly; (2) run a backward logical simulation based on the fold-change data; (3) examine the resulting derived network and choose the most implicated nodes—the ones which are the highest ranking possible causes of the observed data; (4) for that set of nodes, return to the original assembly and run a pathfinding algorithm to find the derived network which is the minimal graph connecting the nodes; and (5) output the resulting derived network as a graph. Methods such as this example can be embodied as functions in the programming framework and can be named and re-used.

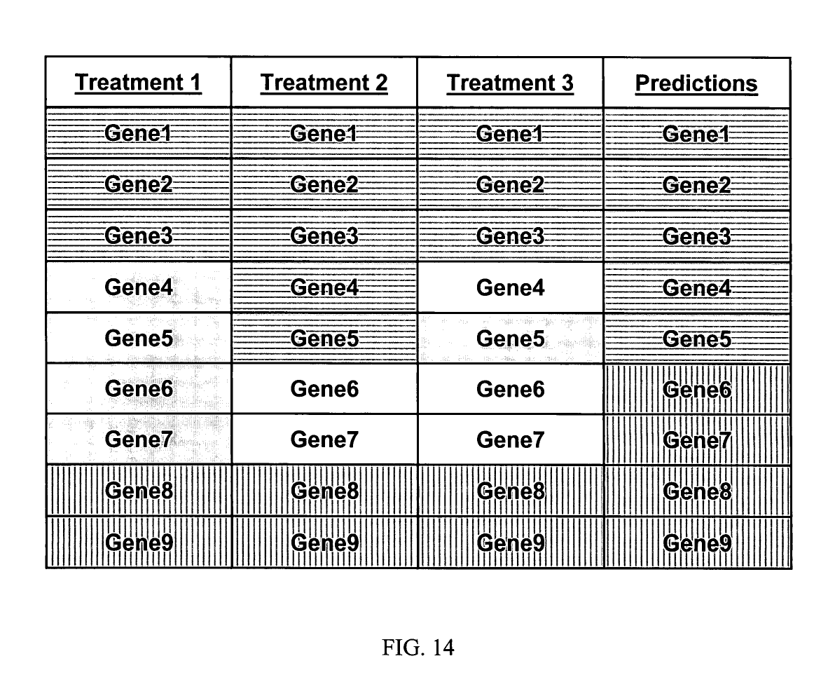

FIG. 14 illustrates a visualization technique comprising an aspect of the present invention that is based on a forward simulation that compares predicted outcomes with actual laboratory data. This diagram shows the direct downstream effects of a perturbation. The right-most column shows the expected outcome of a perturbation in the system. Each predicted value is compared to the actual values to determine how closely the predictions explain the lab data. A correlation can be calculated between the predicted outcome and the actual effect of each treatment. In FIG. 14, the cells marked with horizontal lines show a significant increase, the cells marked with vertical lines show a significant decrease, the darkened cells show no change, and the undarkened cells are insignificant. Perturbations may include, but are not limited to, the increase or decrease in concentration of a transcription factor, a small molecule, or a biochemical catalyst.

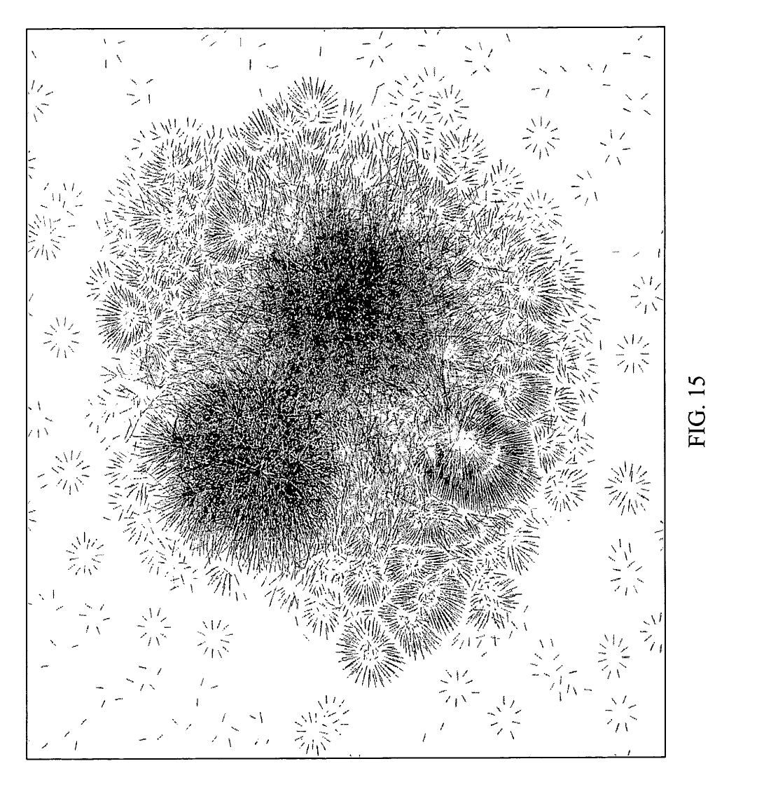

FIG. 15 shows an assembly overview graph, which illustrates the connectivity of the underlying assembly from which it was generated. It can give a biologist a quick visual overview of the number of assertions, the distribution of different types of assertions in the assembly, and the density or degree to which the underlying assembly is connected. The visual overview can be used to determine if the underlying assembly has a sufficient volume of knowledge in a given area, whether the underlying assembly has enough different types of assertions, or whether the underlying assembly has a sufficient density of assertions. Two diagrams representing two different assemblies may be compared side-by-side to determine if one assembly contains more knowledge than the other. One type of comparison would be to compare two diagrams representing the same knowledge base at two different time points to visually inspect the growth of knowledge. The mechanics of generating the diagram of FIG. 15 are as follows: all of the nodes and assertions in the assembly are converted into a diagram by applying a graph layout algorithm to generate a two-dimensional diagram of the assembly. The resulting monochrome diagram shows the scale of the knowledge contained in the assembly and can be used as a starting point for other visualizations. The assembly overview graph can be improved by highlighting assertions containing a particular relationship descriptor with a specific color.

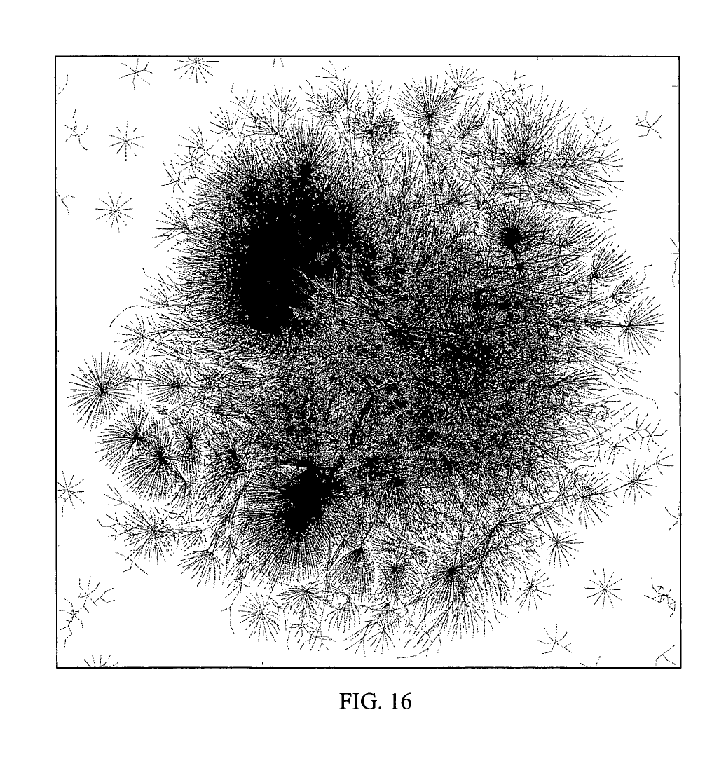

A variation of the assembly overview graph is to generate a graph showing simulation results, as shown in FIG. 16. This diagram can be produced by starting with a monochrome assembly overview graph. The results of a simulation are then overlaid on this diagram. Causal chains of inference can be highlighted by annotating nodes according to their degree of implication. For example, all nodes which are implicated and which the hypothesis predicts are decreased may be annotated by coloring the nodes red, or by replacing the node icon with some other icon, such as a downward pointing arrow. Other node statuses may be indicated by analogous choices of color or icon. The assertions between nodes may also be changed in appearance in order to highlight their causal significance. FIG. 16 shows backward simulation results highlighted in dark gray, and the rest of the assembly is light gray. The graphical output can help a biologist determine the extent of the effects of a given perturbation to the system.

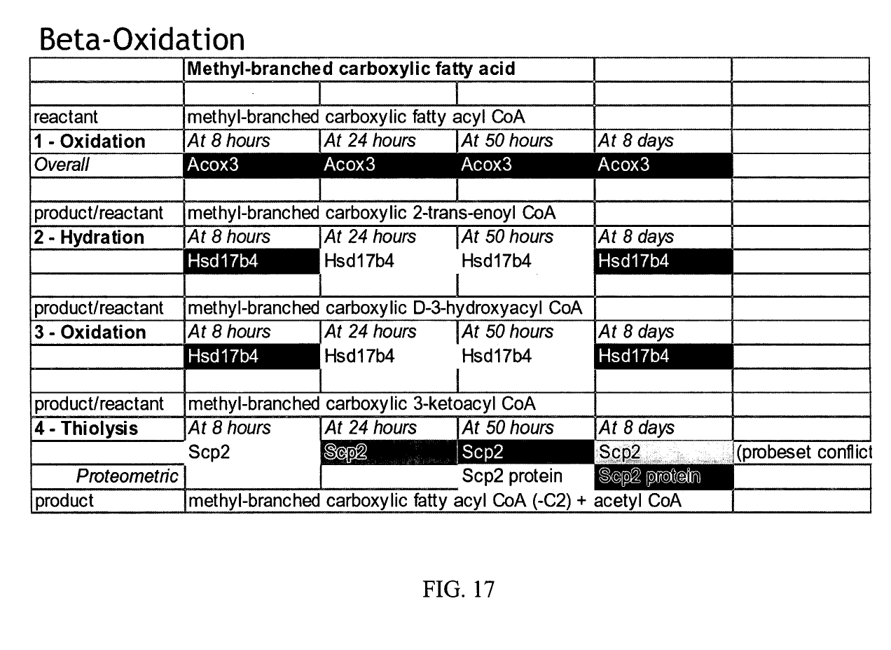

FIG. 17 shows a visualization of time series expression and proteometric data mapped onto a segment of a known metabolic pathway. In some embodiments, background colors may indicate amount and direction of change relative to controls. Each colored cell corresponds to a particular protein, either showing the changes in expression level of its corresponding gene, or the changes in its observed protein abundance. Each column labeled with a time point can indicate data values for a particular experiment in the time series. This method of display is intended to make clear the changes in the modulation of a pathway over a series of experiments, in this case a time-course of treatment. In FIG. 17, shading is used to show expression levels over time (i.e., the darker the shading, the greater the gene expression).

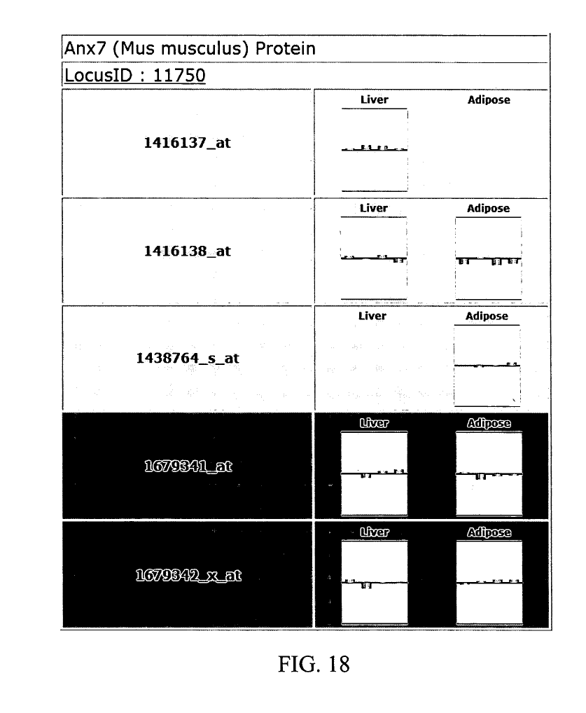

FIG. 18 shows a diagram that indicates a means of summarizing, for a particular gene or protein, time, dose, or other series data from many experiments. One key point is that each horizontal block indicates a particular kind of measurement which can be attributed to the gene or protein. In this example, the protein Anx7 (Mus musculus) is associated with five types of measurements—two are proteometric measurement via 2D gel, three are microarray probe set data yielding gene expression measurements. In this case, the data is expressed as fold changes versus controls, but in other cases it may be desirable to graph absolute values. For each type of measurement, eight fold changes are displayed as histogram bars. In general, any number of data points may be displayed in this manner, up to some practical limit based on the resolution of the display medium. The bars may be color coded—for example, red to show downward changes, and green to show upward changes—in order to make the general trend of each set of measurements more obvious to the user who may be scanning hundreds of these displays when reviewing a dataset. The background colors of each bar may also show the significance of the data. For example, the expression data in the experiment is actually the average of multiple replicates of each experiment, and so a statistical measurement of significance may be assigned to each data point. In one embodiment, a blue background may indicate the most significant data, p-value<0.01, while a magenta background may indicate p-value<0.05. Additionally, a yellow background may indicate any higher p-value. This technique allows the user to easily see the details of the data, details which may have been suppressed in more abstract displays such as a network graph where nodes are simply colored to indicate “up” or “down”, but where those designations are derived from multiple data points.

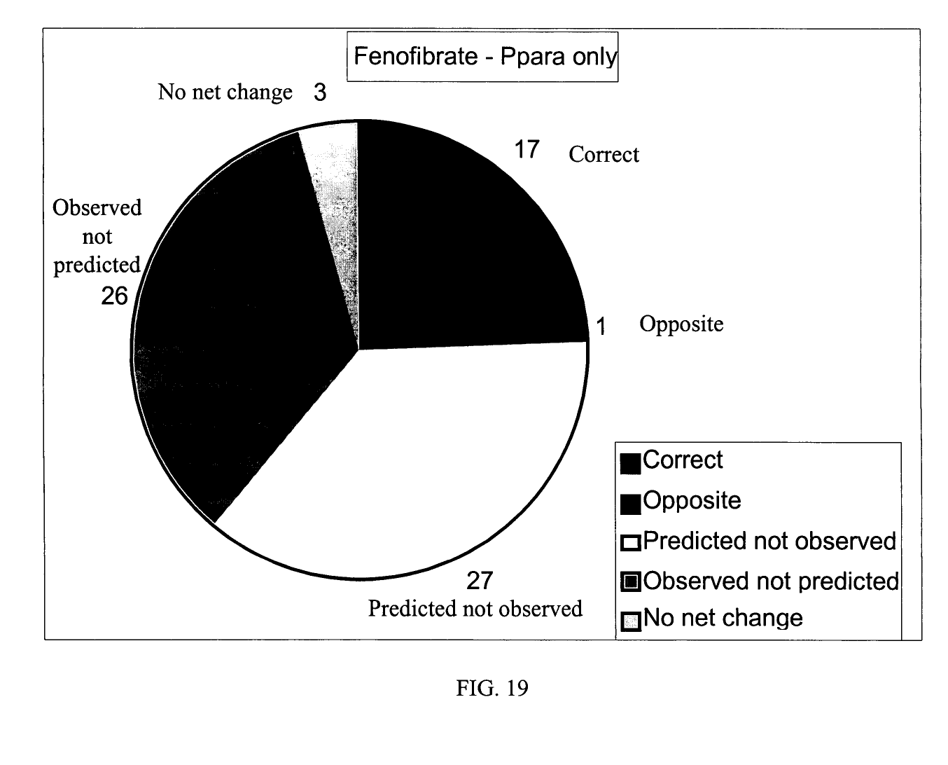

FIG. 19 shows a pie chart that summarizes the correspondence of the changes predicted by a hypothesis to the changes observed in a large dataset. The dataset in this example consists of expression changes due to treatment of hepatocytes with fenofibrate. The hypothesis is that the changes are due to an increase in the activity of the transcription factor PPARA. The pie chart in FIG. 19 displays the following five categories: (1) correct predictions (17%) that are confirmed by the data; (2) opposite predictions (1%) that are contradicted by the data; (3) predictions (27%) that are not observed in the data; (4) data observations (26%) that have no corresponding predictions; and (5) conflicted predictions (3%) for which no net change in the data can be ascribed.

### EXAMPLE 1

Validation Algorithm for Biological Models

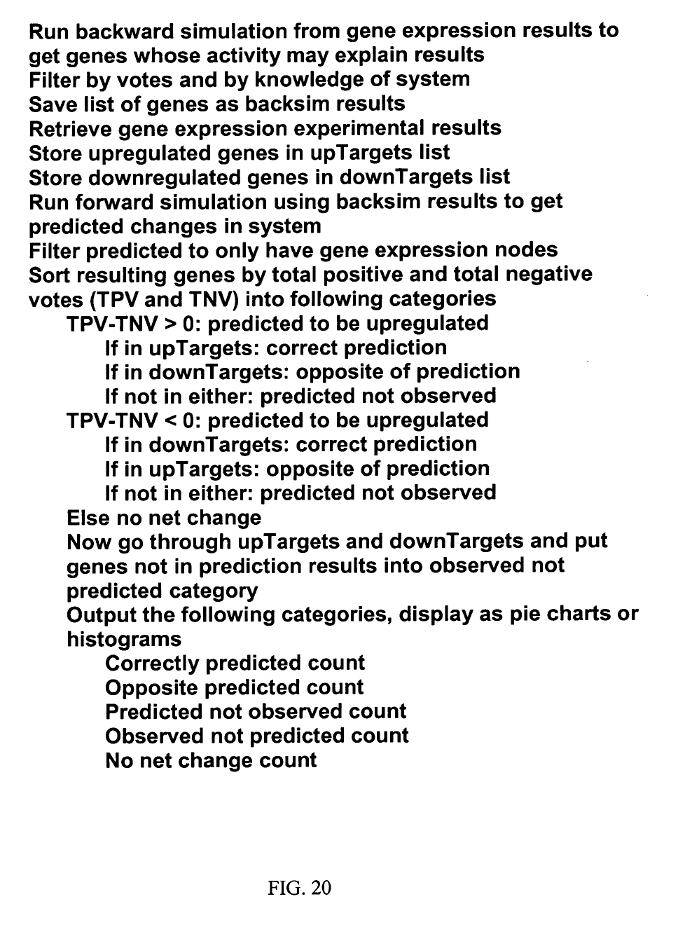

An example of an algorithm for use in validating a biological model by comparing predicted to actual results is described below and in the pseudo code in FIG. 20. This algorithm assumes that there exists a knowledge base representing a biological system with data from gene expression experiments mapped onto the knowledge base.

The predicted results can be determined in two stages. First, a backward simulation as described herein is run on a knowledge base to determine potential causes of the gene expression changes. The backward simulation produces a list of genes and a score for each. The score for each node is based on the “votes” it received during the backward simulation. At the beginning of the backward simulation, nodes representing genes which are significantly upregulated are assigned positive votes, while those which are significantly downregulated are assigned negative votes. During the simulation, votes are copied from node to node according to a set of rules which follow the causal relationships expressed in the knowledge base. At the end of the simulation, the score for each node is computed as a set of three numbers: the sum of positive votes, the sum of negative votes, and an overall score, which is the sum of the positive and negative votes. At this point, the set of nodes representing potential causes (“the causes”) may be used for the next step and may be selected based on each node's score, or the set of potential causes may be determined manually. In the second stage, the votes for all nodes are set to zero and a forward simulation as described herein is run on the selected set of causes. The votes are handled in the same way, except that they are propagated from causes to potential effects. At the end of the forward simulation, nodes which represent the expression of genes are reviewed. Those with a positive overall score are the ones which the forward simulation predicts to be up-regulated and those with a negative overall score are the ones which are predicted to be down-regulated. The results of the forward simulation represent the overall predicted results.

The actual results are classified into two categories based on the gene expression data. One list contains up-regulated genes and another list contains down-regulated genes. The genes included in these lists can be generated by various statistical methods, taking into account the absolute magnitude of the change (e.g., signal level), the relative magnitude of the change (e.g., fold values), statistical significance, etc. Alternatively, the genes may be selected manually.

After the predicted and actual results have been generated, overall results for each gene in the following three cases are tabulated. In the first case, a gene is predicted to be up-regulated. If the gene is in the actual list of up-regulated genes, the “correct prediction counter” is incremented. Otherwise, if the gene is in the actual list of down-regulated genes, the “opposite prediction counter” is incremented. If the gene is not in either list of actual gene expression changes, then the “predicted but not observed counter” is incremented. In the second case, a gene is predicted to be down-regulated. If the gene is in the actual list of up-regulated genes, the “opposite prediction counter” is incremented. Otherwise, if the gene is in the actual list of down-regulated genes, the “correct prediction counter” is incremented. If the gene is not in either list of actual gene expression changes, then the “predicted but not observed counter” is incremented. In the third case, there is no prediction for the gene and the “no net change counter” is incremented.

For every gene that is either in the actual up-regulated or down-regulated gene lists, but does not have any predictions, the “observed not predicted counter” is incremented. The five “counters” are then outputted: (1) “correct prediction counter”, (2) “opposite prediction counter”, (3) “predicted but not observed counter”, (4) “observed not predicted counter”, and (5) “no net change counter”. These counters may be visualized, for example, in a histogram format, or pie chart format, as shown in FIG. 19. Such visualizations provide an intuitive means for a scientist to initially assess the degree to which the generated hypothesis matches the observed data.

### EXAMPLE 2

Biomarker Identification Algorithm

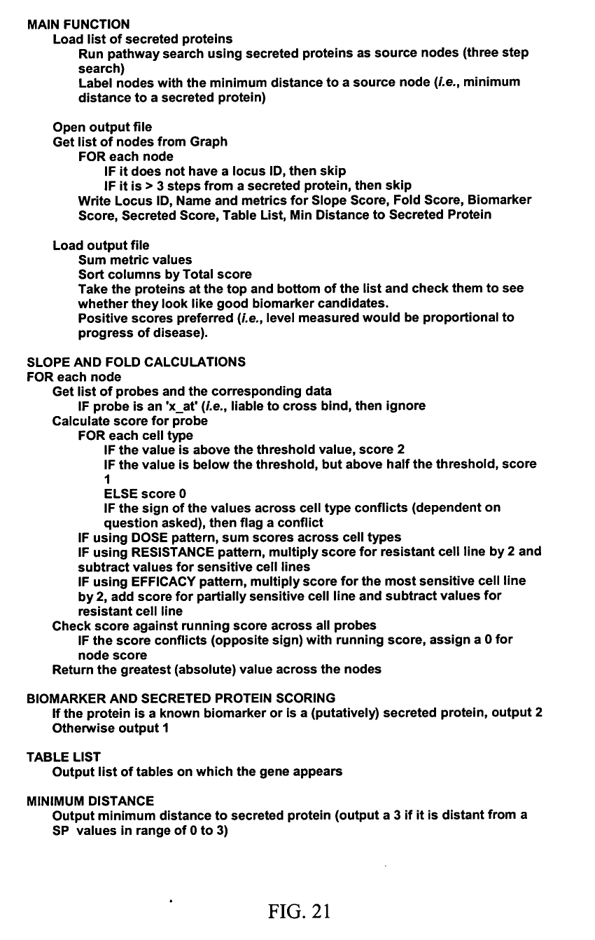

An example of a biomarker identification algorithm in accordance with the invention is described below and in the pseudo code in FIG. 21. In general, this algorithm looks at data characterizing a candidate protein and scores it by taking into account a number of key factors that would make the protein a suitable biomarker. The algorithm brings together metrics from a number of sources, assigns a numerical value, and pools them together to give an overall score which can be used to assess any protein. Specifically, the proteins with the highest absolute score have the greatest number of similarities to an ideal biomarker. The factors used in this example are gene expression changes with a drug, existing knowledge about the nature of the gene product, and proximity to a known biomarker. The algorithm was applied to datasets derived from an experiment in which gene expression changes were measured in response to a drug, across three cell lines of varying susceptibility to this drug.

The first step of the biomarker algorithm is to run a pathway search starting from a list of known secreted proteins. At each step in the search, nodes are labeled with the minimum distance to a source node, i.e. the number of steps away from a secreted protein. The second step is to take the list of proteins that are in the assembly, and iterate through them. For each protein on the list, a list of metrics is calculated as follows: slope and fold calculation, biomarker and secretion score, distance from a secreted protein (calculated in the first step). These metrics are written to a row in an output file. Fold calculations refer to the data expressed as fold changes versus controls, and can be calculated in several ways, for example, (1) disease vs. normal; (2) drug treated vs. non-drug; and (3) resistance vs. susceptibility. Slope is a measure of the rate of change of a series of data points. A data series may be taken, for example at different time points or at different dosage levels. One method to determine the slope of a series is to use linear regression, which results in a straight line that best fits the series of data.

Scores for the slope are measured by looking at the gene expression measurements across three cell types for each probe that corresponds to the protein. Probes that are subject to cross binding are ignored. The remaining values are compared with a reference level, assigning a value of 2 if the slope exceeds this, a value of 1 if it exceeds half the reference level, or 0 if the slope is below half the reference level. For negative slopes, the assigned value is negated. Three patterns are looked for across the cell lines and probe scores calculated according to which one is being used. For a dose-dependent pattern the values across the cell types is summed. For a resistance pattern, the value for the resistant cell line is multiplied by 2 and the values for the two sensitive cell lines is subtracted. For an efficacy pattern, the value for the most sensitive cell line is doubled, the value for the partly sensitive cell line is added and the value for the resistant cell line is subtracted. Scores across the probes are compared and if signs opposed for any pair an overall score of zero is returned to indicate a conflict. In all other cases, the value of the greatest (or most negative) score is returned. Calculations for the fold values are done in the same manner.

For biomarker scoring, a score of 2 is recorded if the protein is a known biomarker, or a score of 1 is recorded if not. Similarly, for secreted proteins, a score of 2 is recorded if it is a (putatively) secreted protein, otherwise record a score of 1 is recorded.

The output file is sorted using an algorithm that calculates an overall score based on the values of the metrics. In the current example, just the fold score is used. Proteins that have the highest absolute values (i.e., those at the top and bottom of the sorted list) are selected for further evaluation as to whether they would by good candidates for biomarkers.

The main components of the score of the algorithm are based on gene expression data. For each locus ID, there are values for multiple probe sets which are processed to give slope and fold change values. The metrics for each locus ID are calculated by pooling the data for the probes, while checking for conflicts of sign (conflicts would result in a 0 score). The algorithm may check for dose dependency, sensitivity, resistance, and efficacy of the drug, and the scoring metric calculates differently for each one. For example, if one is looking at a resistance pattern, it would score slope favorably if the two resistant cell lines were the same and the sensitive cell line differed, whereas the dosage response looked for a paralleled change across all cell lines. The above-detailed algorithm returns a list which is then sorted by column and the genes which rise to the top (fold) are assessed as good potential biomarkers.

While the invention has been particularly shown and described with reference to specific embodiments and illustrative examples, it should be understood by those skilled in the art that various changes in form and detail may be made therein without departing from the spirit and scope of the invention as defined by the appended claims. The scope of the invention is thus indicated by the appended claims and all changes which come within the meaning and range of equivalency of the claims are therefore intended to be embraced.

1. A method of generating new biological knowledge comprising the steps of:
(a) providing a database of biological assertions comprising a multiplicity of nodes representative of biological elements and descriptors characterizing the elements or relationships among nodes;
(b) extracting a subset of assertions from the database that satisfy a set of biological criteria specified by a user to define a selected biological system;
(c) compiling the extracted assertions to produce an assembly comprising a biological knowledge base of assertions potentially relevant to said selected biological system; and
(d) transforming said assembly to generate new biological knowledge about said selected biological system.

2. The method of claim 1, wherein step (d) comprises applying reasoning to said extracted assertions to generate new biological knowledge.

3. The method of claim 1, comprising the additional step of applying reasoning to said extracted assertions to remove logical inconsistencies in said assembly.

4. The method of claim 1, comprising the additional step of applying reasoning to said extracted assertions to augment the assertions therein by adding to said assembly additional assertions from said database.

5. The method of claim 1, comprising the additional step of applying reasoning to said extracted assertions to augment the assertions therein by adding to said database additional assertions that are novel to said assembly.

6. The method of claim 1, comprising the additional step of applying pathway analysis to said knowledge assembly to extract one or more pathways.

7. The method of claim 1, comprising the additional step of applying homology transformation to said extracted assertions.

8. The method of claim 1, comprising the additional step of applying logical simulation to said extracted assertions.

9. The method of claim 1, comprising the additional step of adding to said assembly additional assertions from data sources extraneous to said database.

10. The method of claim 1, wherein said nodes comprise enzymes, cofactors, enzyme substrates, enzyme inhibitors, DNAs, RNAs, transcription regulators, DNA activators, DNA repressors, signaling molecules, trans membrane molecules, transport molecules, sequestering molecules, regulatory molecules, hormones, cytokines, chemokines, antibodies, structural molecules, metabolites, vitamins, toxins, nutrients, minerals, agonists, antagonists, ligands, receptors, or combinations thereof.

11. The method of claim 1, wherein said nodes comprise protons, gas molecules, organic molecules, amino acids, peptides, protein domains, proteins, glycoproteins, nucleotides, oligonucleotides, polysaccharides, lipids, glycolipids, or combinations thereof.

12. The method of claim 1, wherein said nodes comprise cells, tissues, or organs.

13. The method of claim 1, wherein said nodes comprise drug candidate molecules.

14. The method of claim 1, wherein said biological assertions comprise information representative of experimental data, knowledge from the literature, patient data, clinical trial data, compliance data; chemical data, medical data, or hypothesized data.

15. The method of claim 1, wherein said biological assertions comprise information representative of a molecule, biological structure, physiological condition, trait, phenotype, or biological process.

16. An article of manufacture having a computer-readable program carrier with computer-readable instructions embodied thereon for performing the method of claim 1.

17. A method for assembling a biological knowledge base comprising the steps of:
(a) providing a database comprising a multiplicity of nodes representative of biological elements and case frames representative of interrelationships among nodes;
(b) extracting a subset of case frame structures from the database that satisfy a set of biological criteria specified by a user to define a selected biological system; and
(c) compiling the extracted case frame structures to produce an assembly comprising a biological knowledge base of assertions potentially relevant to said selected biological system.

18. A system for assembling a biological knowledge base comprising:
(a) a database of biological assertions comprising a multiplicity of nodes representative of biological elements and descriptors characterizing the elements or relationships among nodes;
(b) an application to extract a subset of assertions from the database that satisfy a set of biological criteria specified by a user to define a selected biological system; and
(c) a knowledge assembler configured to compile the extracted assertions to produce an assembly comprising a biological knowledge base of assertions potentially relevant to said selected biological system.

19. The system of claim 18, further comprising an application for applying reasoning to said extracted assertions to remove logical inconsistencies in said assembly.

20. The system of claim 18, further comprising an application for applying reasoning to said extracted assertions to generate new biological knowledge.

21. The system of claim 18, further comprising an application for applying reasoning to said extracted assertions to augment the assertions therein by adding to said assembly additional assertions from said database satisfying said biological criteria.

22. The system of claim 18, further comprising an application for applying reasoning to said extracted assertions to augment the assertions therein by adding to said knowledge base additional assertions that are novel to said assembly.

23. The system of claim 18, further comprising an application for applying pathway analysis to said knowledge assembly to extract one or more pathways.

24. The system of claim 18, further comprising an application for applying homology transformation to said extracted assertions.

25. The system of claim 18, further comprising an application for applying logical simulation to said extracted assertions.

26. The system of claim 18, further comprising an application for adding to said assembly additional assertions from data sources extraneous to said database.

27. The system of claim 18, wherein said nodes comprise enzymes, cofactors, enzyme substrates, enzyme inhibitors, DNAs, RNAs, transcription regulators, DNA activators, DNA repressors, signaling molecules, trans membrane molecules, transport molecules, sequestering molecules, regulatory molecules, hormones, cytokines, chemokines, antibodies, structural molecules, metabolites, vitamins, toxins, nutrients, minerals, agonists, antagonists, ligands, receptors, or combinations thereof.

28. The system of claim 18, wherein said nodes comprise protons, gas molecules, organic molecules, amino acids, peptides, protein domains, proteins, glycoproteins, nucleotides, oligonucleotides, polysaccharides, lipids, glycolipids, or combinations thereof.

29. The system of claim 18, wherein said nodes comprise cells, tissues, or organs.

30. The system of claim 18, wherein said nodes comprise drug candidate molecules.

31. The system of claim 18, wherein said biological assertions comprise information representative of experimental data, knowledge from the literature, patient data, clinical trial data, compliance data; chemical data, medical data, or hypothesized data.

32. The system of claim 18, wherein said biological assertions comprise information representative of a molecule, biological structure, physiological condition, trait, phenotype, or biological process.

33. An article of manufacture having a computer-readable program carrier with computer-readable instructions embodied thereon for using the system of claim 18.

34. A computing device for assembling a biological knowledge base comprising:
(a) means for accessing an electronic database of biological assertions comprising a multiplicity of nodes representative of biological elements and descriptors characterizing the elements or relationships among nodes;
(b) a user addressable computer application to extract a subset of assertions from the database that satisfy a set of biological criteria specified by a user to define a selected biological system; and
(c) a knowledge assembler configured to compile the extracted assertions to produce an assembly comprising a biological knowledge base of assertions potentially relevant to said selected biological system.

35. The computing device of claim 30, further comprising a computer application for applying reasoning to said extracted assertions to remove logical inconsistencies in said assembly.

36. The computing device of claim 30, further comprising a computer application for applying reasoning to said extracted assertions to generate new biological knowledge.

37. The computing device of claim 30, further comprising a computer application for applying reasoning to said extracted assertions to augment the assertions therein by adding to said assembly additional assertions from said database satisfying said biological criteria.

38. The computing device of claim 30, further comprising a computer application for applying reasoning to said extracted assertions to augment the assertions therein by adding to said knowledge base additional assertions that are novel to said assembly.

39. The computing device of claim 30, further comprising a computer application for applying pathway analysis to said knowledge assembly to extract one or more pathways.

40. The computing device of claim 30, further comprising a computer application for applying homology transformation to said extracted assertions.

41. The computing device of claim 30, further comprising a computer application for applying logical simulation to said extracted assertions.

42. The computing device of claim 30, further comprising a computer application for adding to said assembly additional assertions from data sources extraneous to said database.

43. The computing device of claim 30, wherein said nodes comprise enzymes, cofactors, enzyme substrates, enzyme inhibitors, DNAs, RNAs, transcription regulators, DNA activators, DNA repressors, signaling molecules, trans membrane molecules, transport molecules, sequestering molecules, regulatory molecules, hormones, cytokines, chemokines, antibodies, structural molecules, metabolites, vitamins, toxins, nutrients, minerals, agonists, antagonists, ligands, receptors, or combinations thereof.

44. The computing device of claim 30, wherein said nodes comprise protons, gas molecules, organic molecules, amino acids, peptides, protein domains, proteins, glycoproteins, nucleotides, oligonucleotides, polysaccharides, lipids, glycolipids, or combinations thereof.

45. The computing device of claim 30, wherein said nodes comprise cells, tissues, or organs.

46. The computing device of claim 30, wherein said nodes comprise drug candidate molecules.

47. The computing device of claim 30, wherein said biological assertions comprise information representative of experimental data, knowledge from the literature, patient data, clinical trial data, compliance data; chemical data, medical data, or hypothesized data.

48. The computing device of claim 30, wherein said biological assertions comprise information representative of a molecule, biological structure, physiological condition, trait, phenotype, or biological process.

49. A method of discovering new biological knowledge comprising the steps of:
(a) providing a database of biological assertions comprising a multiplicity of nodes representative of biological elements and descriptors characterizing the elements or relationships among nodes;
(b) extracting a subset of assertions from the database that satisfy a set of biological criteria specified by a user to define a selected biological system;
(c) compiling the extracted assertions to produce an assembly comprising a biological knowledge base of assertions potentially relevant to said selected biological system; and
(d) analyzing said assembly to discover new biological knowledge.

50. The method of claim 49, wherein said new biological knowledge comprises predictions of physiological behavior in humans from analysis of experiments conducted on animals.

51. The method of claim 50, wherein said physiological behavior comprises drug efficacy or toxicity.

52. The method of claim 50, wherein said new biological knowledge comprises discovery of a biomarker.

53. The method of claim 49, wherein step (d) comprises repeating said method using a different set of biological criteria to produce a different assembly and comparing different assemblies.

54. The method of claim 49, wherein step (d) comprises mapping experimental data onto an assembly to produce a graphical output.

55. The method of claim 49, further comprising adding putative assertions, distinguished by attribution or a lower trust value, to the knowledge base to allow more speculative results to be produced in step (d).

56. The method of claim 49, wherein step (d) comprises applying pathway analysis to said knowledge assembly to further extract one or more pathways.

57. The method of claim 49, wherein step (d) comprises applying algorithms for mechanism determination.

58. The method of claim 49, wherein step (d) comprises applying visualization techniques to display knowledge and associated data to enhance user understanding and recognition of patterns and clusters.

59. The method of claim 49, comprising the additional step of applying reasoning to said extracted assertions to remove logical inconsistencies in said assembly.

60. The method of claim 49, comprising the additional step of applying reasoning to said extracted assertions to augment the assertions therein by adding to said assembly additional assertions from said database

61. The method of claim 49, comprising the additional step of applying reasoning to said extracted assertions to augment the assertions therein by adding to said knowledge base additional assertions that are novel to said assembly.

62. The method of claim 49, comprising the additional step of applying pathway analysis to said knowledge assembly to extract one or more pathways.

63. The method of claim 49, comprising the additional step of applying homology transformation to said extracted assertions.

64. The method of claim 49, comprising the additional step of applying the results of logical simulation to said extracted assertions.

65. The method of claim 49, comprising the additional step of adding to said assembly additional assertions from data sources extraneous to said database.

66. The method of claim 49, wherein said nodes comprise enzymes, cofactors, enzyme substrates, enzyme inhibitors, DNAs, RNAs, transcription regulators, DNA activators, DNA repressors, signaling molecules, trans membrane molecules, transport molecules, sequestering molecules, regulatory molecules, hormones, cytokines, chemokines, antibodies, structural molecules, metabolites, vitamins, toxins, nutrients, minerals, agonists, antagonists, ligands, receptors, or combinations thereof.

67. The method of claim 49, wherein said nodes comprise protons, gas molecules, organic molecules, amino acids, peptides, protein domains, proteins, glycoproteins, nucleotides, oligonucleotides, polysaccharides, lipids, glycolipids, or combinations thereof.

68. The method of claim 49, wherein said nodes comprise cells, tissues, or organs.

69. The method of claim 49, wherein said nodes comprise drug candidate molecules.

70. The method of claim 49, wherein said biological assertions comprise information representative of experimental data, knowledge from the literature, patient data, clinical trial data, compliance data; chemical data, medical data, or hypothesized data.

71. The method of claim 49, wherein said biological assertions comprise information representative of a molecule, biological structure, physiological condition, trait, phenotype, or biological process.

72. An article of manufacture having a computer-readable program carrier with computer-readable instructions embodied thereon for performing the method of claim 49.

73. The method of claim 49, comprising the additional steps of generating a hypothesis concerning a pathway based on said new biological knowledge, and conducting a biological experiment using biomolecules, cells, animal models, or a clinical trial to validate or refute said hypothesis.

74. A method of generating new biological knowledge comprising the steps of:
(a) providing a database of biological assertions comprising a multiplicity of nodes representative of biological elements and descriptors characterizing the elements or relationships among nodes; and
(b) transforming a plurality of said biological assertions to produce an assembly comprising a derived knowledge network.

75. The method of claim 74, wherein said transforming step comprises inferring new assertions from said biological assertions.

76. The method of claim 75, wherein said new assertions comprise a multiplicity of new nodes representative of new biological elements and descriptors characterizing the elements or relationships among new nodes.

77. The method of claim 74, wherein said transforming step comprises extracting a subset of assertions from the database that satisfy a set of biological criteria specified by a user to define a selected biological system.

78. The method of claim 74, wherein said transforming step comprises performing mathematical set operations on said biological assertions to produce new assertions.

79. The method of claim 78, wherein said new assertions comprise a multiplicity of new nodes representative of new biological elements and descriptors characterizing the elements or relationships among new nodes.

80. The method of claim 74, wherein said transforming step comprises summarizing said biological assertions to produce new assertions.

81. The method of claim 80, wherein said new assertions comprise a multiplicity of new nodes representative of new biological elements and descriptors characterizing the elements or relationships among new nodes.

82. A system for assembling a biological knowledge base comprising:
(a) a database of biological assertions comprising a multiplicity of nodes representative of biological elements and descriptors characterizing the elements or relationships among nodes; and
(b) an application to transform a plurality of said biological assertions to produce an assembly comprising a derived knowledge network.

83. The system of claim 82, wherein said application infers new assertions from said biological assertions.

84. The system of claim 83, wherein said new assertions comprise a multiplicity of new nodes representative of new biological elements and descriptors characterizing the elements or relationships among new nodes.

85. The system of claim 82, wherein said application extracts a subset of assertions from the database that satisfy a set of biological criteria specified by a user to define a selected biological system.

86. The system of claim 85, wherein said extracted assertions comprise a multiplicity of new nodes representative of new biological elements and descriptors characterizing the elements or relationships among new nodes.

87. The system of claim 82, wherein said application performs mathematical set operations on said biological assertions to produce new assertions.

88. The system of claim 87, wherein said new assertions comprise a multiplicity of new nodes representative of new biological elements and descriptors characterizing the elements or relationships among new nodes.

89. The system of claim 82, wherein said application summarizes said biological assertions to produce new assertions.

90. The system of claim 89, wherein said new assertions comprise a multiplicity of new nodes representative of new biological elements and descriptors characterizing the elements or relationships among new nodes.

91. A method of mining a biological knowledge database comprising:
(a) providing a database of biological assertions comprising a multiplicity of nodes representative of biological elements and descriptors characterizing the elements or relationships among nodes;
(b) transforming a plurality of said biological assertions to produce an assembly comprising a derived knowledge network; and
(c) mining said assembly to discover new biological knowledge.

92. The method of claim 92, wherein step (c) comprises mapping experimental data onto an said assembly to produce a graphical output.

93. The method of claim 91, further comprising adding putative assertions, distinguished by attribution or a lower trust value, to the knowledge base to allow more speculative results to be produced in step (c).

94. The method of claim 91, wherein step (c) comprises applying pathway analysis to said assembly to produce one or more pathways that relates to experimental data or clinical data of interest.

95. The method of claim 91, wherein step (c) comprises applying algorithms for mechanism determination.

96. The method of claim 91, wherein step (c) comprises applying visualization techniques to display knowledge and associated data to enhance user understanding and recognition of patterns and clusters.

97. The method of claim 91, comprising the additional step of applying reasoning to said assertions to augment the assertions therein by adding to said knowledge base additional assertions that are novel to said assembly.

98. The method of claim 91, comprising the additional step of applying pathway analysis to said assembly to produce one or more pathways that relates to experimental data or clinical data.

99. The method of claim 91, comprising the additional step of applying homology transformation to said assertions.

100. The method of claim 91, comprising the additional step of applying the results of logical simulation to said assertions.

101. The method of claim 91, comprising the additional step of adding to said assembly additional assertions from data sources extraneous to said database.

102. A method for assembling a biological knowledge base comprising the steps of:
(a) providing a database of biological assertions comprising a multiplicity of nodes representative of biological elements and descriptors characterizing the elements or relationships among nodes;
(b) extracting a subset of assertions from the database that satisfy a set of biological criteria specified by a user to define a selected biological system;
(c) compiling the extracted assertions to produce an assembly comprising a biological knowledge base of assertions potentially relevant to said selected biological system; and
(d) altering the granularity of said assembly by:
(i) removing individual nodes or sub networks of nodes from said assembly while retaining within the assembly relationships between nodes not directly linked by relationship descriptors in said database;
(ii) merging plural pathways defined by linked nodes;
(iii) collapsing nodes;
(iv) summarizing a sub assembly of the assembly as a new node; or
(v) adding nodes from the database further elaborating a link between a pair of nodes in the assembly.

103. A method for generating new biological knowledge comprising the steps of:
(a) providing a database of biological assertions comprising a multiplicity of nodes representative of biological elements and descriptors characterizing the elements or relationships among nodes;
(b) extracting plural subsets of assertions from the database that satisfy sets of biological criteria specified by a user to define plural sub databases relevant to selected biological systems;
(c) compiling the extracted assertions to produce at least two biological knowledge base assemblies of assertions potentially relevant to said selected biological systems; and
(d) overlaying said assemblies to discern differences or commonalities between the respective assemblies or merging the assemblies.
